(19) United States
(12) Patent Application Publication
(10) Pub. No.: US 2005/0249663 A1
(43) Pub. Date: Nov. 10, 2005

(54) Coadministration of transport protein with conjugated cobalamin to deliver agents

(75) Inventor: Douglas A. Collins, Rochester, MN (US)

Correspondence Address: KING & SPALDING LLP, 191 PEACHTREE STREET, N.E., 45TH FLOOR, ATLANTA, GA, 30303-1763, US

(73) Assignee: Mayo Foundation for Medical Education and Research, Rochester, MN (US)

(21) Appl. No.: 10/918,638

(22) Filed: Aug. 12, 2004

Related U.S. Application Data
(63) Continuation of application No. 10/262,318, filed on Sep. 30, 2002, now abandoned.
(60) Provisional application No. 60/326,183, filed on Sep. 28, 2001.

(51) Int. Cl. A61K 51/00; A61K 49/00; A61K 48/00

(52) U.S. Cl. 424/1.11; 424/9.362; 514/44; 514/52; 514/81

(57) ABSTRACT

Cobalamin transport proteins are administered in combination with cobalamin coupled to a diagnostic or pharmaceutically active agents to increase the extent of absorption of the diagnostic or pharmaceutically active agent. Cobalamin transport proteins include, but are not limited to intrinsic factor, transcobalamin I, transcobalamin II and transcobalamin III. The combination of the cobalamin or cobalamin derivative with the cobalamin transport protein provides enhanced cellular uptake.

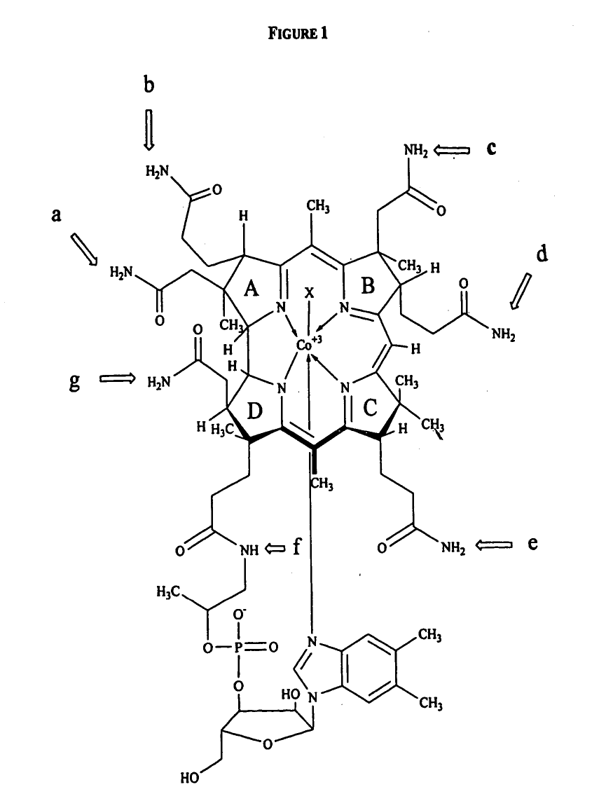

This application claims priority to U.S. Ser. No. 60/326,183 filed on Sep. 28, 2001.

### FIELD OF THE INVENTION

This invention is the coadministration of cobalamin or a derivative thereof linked to a diagnostic or therapeutic agent with a transport protein to increase the amount of agent delivered to the host cell.

### BACKGROUND OF THE INVENTION

Vitamin B₁₂ is critical to normal physiological functioning. Vitamin B₁₂ is water soluble, has no known toxicity and in excess is excreted by glomerular filtration. B₁₂ participates in at least two essential intracellular metabolic pathways. For several years after the isolation of vitamin B₁₂ as cyanocobalamin in 1948, it was assumed that cyanocobalamin and possibly hydroxocobalamin, its photolytic breakdown product, occurred in man. Since then it has been recognized that cyanocobalamin is an artifact of the isolation of vitamin B₁₂ and that hydroxycobalamin and the two coenzyme forms, methylcobalamin and adenosylcobalamin, are the naturally occurring materials in the body. The derivative methylcobalamin serves as the cofactor for methionine synthetase, which catalyzes the methylation of homocysteine in which N⁵-methyl-tetrahydrofolate provides the methyl groups and tetrahydrofolate becomes available for recycling in the various folate pathways. Deoxyadenosylcobalamin functions with methylmalonyl CoA mutase in a metabolic pathway in the rearrangement of methylmalonyl-CoA to succinylCoA. B₁₂ cannot be synthesized by higher organisms.

The physiological utilization of B₁₂ or a biological metabolite requires the interface of a number of intricately woven mechanisms requiring binding proteins and membrane receptors for absorption, transport and cellular uptake. The most important of these proteins include: intrinsic factor (IF), a protein secreted by gastric parietal cells that binds dietary B₁₂ and transports further along to the ileum for absorption; an IF-B₁₂ receptor on the brush borders of the epithelial mucosa in the mid- to terminal ileum that binds and internalizes IF-B₁₂; transcobalamin II (TCII), a plasma protein that transports B₁₂ from the site of intestinal absorption and from its primary storage site, the liver, to bodily tissues, and specific receptors on the plasma membrane of tissue cells than bind and internalize the TCII-B₁₂ complex.

Vitamin B₁₂ (adenosyl-, cyano-, hydroxo-, or methylcobalamin) must be bound by the transport protein Transcobalamin I, II, or III (“TC”) to be biologically active, and by IF if administered orally. Gastrointestinal absorption of vitamin B₁₂ occurs when the IF-B₁₂ complex is bound to the IF-B₁₂ receptor in the terminal ileum. Likewise, intravascular transport and subsequent cellular uptake of vitamin B₁₂ throughout the body typically occurs through the cobalamin transport protein (I, I or III) and the cell membrane cobalamin receptors, respectively. After the cobalamin transport protein-vitamin B₁₂ complex has been internalized in the cell, the transport protein undergoes lysozymal degradation, which releases vitamin B₁₂ into the cytoplasm. All forms of vitamin B₁₂ can then be interconverted into adenosyl-, hydroxo- or methylcobalamin depending upon cellular demand. See, for example, A. E. Finkler et al., Arch. Biochem. Biophys., 120, 79 (1967); C. Hall et al., J. Cell Physiol., 133, 187 (1987); M. E. Rappazzo et al., J. Clin. Invest., 51, 1915 (1972) and R. Soda et al., Blood, 65, 795 (1985).

Both IF and TC-II deficiencies lead to abnormalities such as megaloblastic anemia and demyelinating disorders of the nervous system. In plasma, TC-I turns over very slowly (t_{1/2}=10 days) and appears to serve as the major storage protein for cobalamin. Allen (1975) has suggested that TC-I may participate in the storage of excess cobalamin and bind degraded cobalamin for removal. TC-I may also stabilize serum cobalamin against transdermal photolysis.

Once IF-B₁₂ is attached to the apical brush border membrane, there is a 3-4-hour delay before B₁₂ exits the enterocyte bound to TC. IF-B₁₂ is internalized via receptor-mediated endocytosis (Seetharam et al., 1985). The process of transcytosis is typically in the 24-48 hours range.

The receptor for IF-B₁₂ has been purified and has been designated cubilin. Cubilin has no apparent homology to other known receptors. The binding of IF-B₁₂ to cubilin is a high-affinity interaction, with a single binding site (K_{d}=1-5 nM at 20° C.) dependent on calcium.

The primary function of TC-II is to deliver B₁₂ to the tissues following intestinal absorption of the vitamin. The liver, the major storage site for B₁₂, is a source from which B₁₂ can be transferred to TC-II under conditions of dietary deficiency (e.g., a strict vegetarian diet) or when an individual is unable to absorb the vitamin from the diet (e.g. pernicious anemia, surgical removal of the stomach or distal small intestine, malabsorption). Clearance of TC-II bound to B₁₂ from the plasma is rapid.

TC-II bound to B₁₂ in plasma is carried to cells expressing the TC-II receptor on the plasma membrane, which binds and internalizes the complex by endocytosis. The TC-II/B₁₂/receptor complex is processed in the endosome with dissociation of the receptor and TC-II bound to B₁₂. Following lysosomal fusion, B₁₂ dissociates from TC-II. The free B₁₂ enters the cytoplasmic and mitochondrial compartments, where the cofactors Me-cobalamin and Ado-cobalamin, respectively, are synthesized.

In plasma and nonintestinal tissue fluids, B₁₂ is bound to a TC. TC-B₁₂, is the essential carrier for the transport of B₁₂ to tissues.

Vitamin B₁₂ derivatives have been proposed as a means to deliver various pharmacotherapeutic agents. Such agents include antibiotics, anti-tumor agents, radiolabels, cardiovascular agents, nutriceuticals and agents useful for the treatment of cellular proliferative disorders.

Processes for preparing derivatives of B₁₂ are known in the art. For example, a process for preparing ¹²⁵I-vitamin B₁₂ derivatives is described in Niswender et al. (U.S. Pat. No. 3,981,863). In this process, vitamin B₁₂ is first subjected to mild hydrolysis to form a mixture of monocarboxylic acids, which Houts, infra, disclosed to contain mostly the (e)-isomer. The mixture is then reacted with a p-(aminoalkyl)phenol to introduce a phenol group into the B₁₂ acids (via reaction with one of the free carboxylic acid groups). The mixed substituent B₁₂ derivatives are then iodinated in the phenol-group substituent. This U.S. patent teaches that the mixed ¹²⁵I-B₁₂ derivatives so made are useful in the radioimmunoassay of B₁₂, using antibodies raised against the mixture.

T. M. Houts (U.S. Pat. No. 4,465,775) reported that the components of the radiolabelled mixture of Niswender et al. did not bind with equal affinity to IF. Houts disclosed that radioiodinated derivatives of the pure monocarboxylic (d)-isomer are useful in assays of B₁₂ in which IF is used.

U.S. Pat. Nos. 5,739,313; 6,004,533; 6,096,290 and PCT Publication WO 97/18231 listing Collins and Hogenkamp as inventors disclose radionuclide labeling of vitamin B₁₂ through the propionamide moieties on naturally occurring vitamin B₁₂. The inventors converted the propionamide moieties at the b-, d-, and e-positions of the corrole ring to monocarboxylic acids, through a mild hydrolysis, and separated the carboxylic acids by column chromatography. The inventors then attached a bifunctional linking moiety to the carboxylate function through an amide linkage, and a chelating agent to the linking moiety again through an amide linkage. The chelating moiety was then used to attach a radionuclide to the vitamin that can be used for therapeutic or diagnostic purposes.

Collins, et al. in WO 01/28595 (PCT/US00/10098) disclose a series of novel cobalamin conjugates that are linked via a protein linker to a detectable group, which are useful in the imaging of tumors.

Collins, et al. in WO 01/28592 (PCT/US00/10097) disclose a series of novel cobalamin conjugates that are linked directly or by a linker to a residue of a chemotherapeutic agents, which are useful in the treatment of abnormal cellular proliferation.

Collins, et al. in WO 00/62808 (PCT/US00/10100) disclose a series of novel cobalamin conjugates that are linked directly or by a linker to a residue of a molecule comprising B-10 or Gd-157, which are useful in the treatment of abnormal cellular proliferation.

PCT Publication WO 98/08859 listing Grissom et al as inventors discloses conjugates containing a bioactive agent and an organocobalt complex in which the bioactive agent is covalently bound directly or indirectly, via a spacer, to the cobalt atom. The organocobalt complex can be cobalamin and the bioactive agent can be a chemotherapeutic agent. However, only one bioactive agent (i.e., chemotherapeutic agent) is attached to the organocobalt complex (i.e., cobalamin) and the attachment is solely through the cobalt atom (i.e., the 6-position of cobalamin). The bioactive agent is released from the bioconjugate by the cleavage of the weak covalent bond between the bioactive agent and the cobalt atom as a result of normal displacement by cellular nucleophiles or enzymatic action, or by application of an external signal (e.g., light, photoexcitation, ultrasound, or the presence of a magnetic field).

U.S. Pat. No. 5,428,023 to Russell-Jones et al. discloses a vitamin B₁₂ conjugate for delivering oral hormone formulations. Russell-Jones teaches that the vitamin B₁₂ conjugate must be capable of binding in vivo to intrinsic factor, enabling uptake and transport of the complex from the intestinal lumen of a vertebrate host to the systemic circulation of the host. The hormones are attached to the vitamin B₁₂ through a hydrolyzed propionamide linkage on the vitamin. The patent states that the method is useful for orally administering hormones, bioactive peptides, therapeutic agents, antigens, and haptens, and lists as therapeutic agents neomycin, salbutamol cloridine, pyrimethamine, penicillin G, methicillin, carbenicillin, pethidine, xylazine, ketamine hydrochloride, mephanesin and iron dextran. U.S. Pat. No. 5,548,064 to Russell-Jones et al. discloses a vitamin B₁₂ conjugate for delivering erythropoietin and granulocyte-colony stimulating factor, using the same approach as the '023 patent.

PCT Publication WO 94/27641 to Russell-Jones et al discloses vitamin B₁₂ linked through a polymer to various active agents wherein the conjugate is capable of binding to intrinsic factor for systemic delivery. In particular, the document discloses the attachment of various polymeric linkers to the propionamide positions of the vitamin B₁₂ molecule, and the attachment of various bioactive agents to the polymeric linker. Exemplary bioactive agents include hormones, bioactive peptides and polypeptides, antitumor agents, antibiotics, antipyretics, analgesics, antiinflammatories, and haemostatic agents. Exemplary polymers include carbohydrates and branched chain amino acid polymers. The linkers used in WO 94/27641 are polymeric (each having a molecular weight of about 5000 or greater). Importantly, the linkers are described as exhibiting a mixture of molecular weights, due to the polymerization process by which they are made. See in particular, page 11, lines 25-26 wherein it is stated that the polymer used in that invention is of uncertain size and/or structure.

PCT Publication WO 99/65930 to Russell-Jones et al. discloses the attachment of various agents to the 5′-OH position on the vitamin B₁₂ ribose ring. The publication indicates that the system can be used to attach polymers, nanoparticles, therapeutic agents, proteins and peptides to the vitamin.

U.S. Pat. No. 5,574,018 to Habberfield et al. discloses conjugates of vitamin B₁₂ in which a therapeutically useful protein is attached to the primary hydroxyl site of the ribose moiety. The patent lists erythropoietin, granulocyte-colony stimulating factor and human intrinsic factor as therapeutically useful proteins, and indicates that the conjugates are particularly well adapted for oral administration.

U.S. Pat. No. 5,840,880 to Morgan, Jr. et al. discloses vitamin B₁₂ conjugates to which are linked receptor modulating agents, which affect receptor trafficking pathways that govern the cellular uptake and metabolism of vitamin B₁₂. The receptor modulating agents are linked to the vitamin at the b-, d; or e-position.

Other patent filings which describe uses of Vitamin B₁₂ include U.S. Pat. No. 3,936,440 to Nath (Method of Labeling Complex Metal Chelates with Radioactive Metal Isotopes); U.S. Pat. No. 4,209,614 to Bernstein et al., (Vitamin B₁₂ Derivatives Suitable for Radiolabeling); U.S. Pat. No. 4,279,859 (Simultaneous Radioassay of Folate and Vitamin B₁₂); U.S. Pat. No. 4,283,342 to Yollees (Anticancer Agents and Methods of Manufacture); U.S. Pat. No. 4,301,140 to Frank et al (Radiopharmaceutical Method for Monitoring Kidneys); U.S. Pat. No. 4,465,775 to Houts (Vitamin B₁₂ and labeled Derivatives for Such Assay); U.S. Pat. No. 5,308,606 to Wilson et al (Method of Treating and/or Diagnosing Soft Tissue Tumors); U.S. Pat. No. 5,405,839 (Vitamin B₁₂ Derivative, Preparation Process Thereof, and Use Thereof); U.S. Pat. No. 5,449,720 to Russell-Jones et al., (Amplification of the Vitamin B₁₂ Uptake System Using Polymers); U.S. Pat. No. 5,589,463 to Russell Jones (Oral Delivery of Biologically Active Substances Bound to Vitamin B₁₂); U.S. Pat. No. 5,608,060 to Axworthy et al (Biotinidase-Resistant Biotin-DOTA Conjugates); U.S. Pat. No. 5,807,832 to Russell-Jones et al (Oral Delivery of Biologically Active Substances Bound to Vitamin B₁₂); U.S. Pat. No. 5,869,465 to Morgan et al (Method of Receptor Modulation and Uses Therefor); U.S. Pat. No. 5,869,466 to Russell-Jones et al (vitamin B₁₂ Mediated Oral Delivery systems for GCSF).

See also Ruma Banerjee, Chemistry and Biochemistry of B₁₂ John Wiley & Sons, Inc. (1999), and in particular Part II, Section 15 of that book, entitled “Diagnostics and Therapeutic Analogues of Cobalamin,” by H.P.C. Hogenkamp, Douglas A. Collins, Charles B. Grissom, and Frederick G. West.

Administration of B₁₂ or B₁₂ conjugated agents suffers from a number of problems. The uptake of B₁₂ into the gastrointestinal system after oral administration is limited by the amount and availability of IF. Only two to five micrograms of B₁₂ can be taken up in the gastrointestinal tract daily, and the percentage of the two to five micrograms that is actually absorbed into the blood stream remains unknown. If B₁₂ or a B₁₂ conjugated agent is administered intravenously, slightly less than one milligram can be absorbed. Typically, twenty five to forty percent is excreted, and the remaining is stored. Deficiencies in IF lead to impaired uptake of B₁₂ and can contribute to disease states as pericious anemia. There is evidence that the uptake of conjugated-B₁₂ is not significantly different from that of unconjugated B₁₂ alone.

Cooper, B A et al ((1961) Nature. 191:393-395) describe that radiolabeled vitamin B₁₂ bound to intrinsic factor showed increased uptake in vitro in human and mouse tumor cells.

Uchino, Haruto et al. ((Apr. 24, 1964) Annals of the NY Academy of Science. 112:844-863) disclose that oral and intravenous administration of adenosyl cobalamin prebound to intrinsic factor in rats resulted in enhanced uptake of the cobalamin in rat tissues. In the forty years since Cooper and Uchino published these preliminary results, and despite concentrated research in the area of cobalamins, there has been negligible pursuit of this line of investigation.

U.S. Pat. No. 6,183,723 to Seetharam et al. discloses a method to treat an intrinsic factor or intrinsic factor receptor deficient patient by conjugating transcobalamin-II to cobalamin. Seetharam et al. discovered a novel pathway by which cobalamin can be absorbed from the gastrointestinal tract through conjugation to transcobalamin II via the transcobalamin II receptor. They disclose that under normal conditions, it is highly unlikely that this transcobalamin II mediated transport bypasses the well accepted intrinsic factor/intrinsic factor receptor mediated cobalamin transport in the gastrointestinal tract, but despite its lack of importance in the normal uptake of cobalamin, it may be useful in patients with inherited disorders such as intrinsic factor or intrinsic factor receptor deficient patients.

It is an object of the invention to provide a method and composition for the increased efficiency of vitamin B₁₂ or vitamin B₁₂ conjugated materials for therapeutic and diagnostic purposes.

### SUMMARY OF THE INVENTION

It has been discovered that the uptake of a cobalamin linked to a diagnostic or therapeutic agent can be significantly enhanced by administering the cobalamin in (covalent, ionic or admixed) combination with a cobalamin transport protein. In a broader embodiment, the amount delivered to cells of any transcobalamin II or intrinsic factor receptor ligand conjugated to a detectable or therapeutic agent can be increased by combination (covalent, ionic or admixed) with a cobalamin transport protein.

Up to this point, it has generally been accepted that there is limited absorption of cobalamin from the gastrointestinal tract (2-5 micrograms per day) as well as through intravenous injection (1 milligram per day). The discovery that the combination of cobalamin transport proteins (such as intrinsic factor, transcobalamin I, transcobalamin II, and transcobalamin III) with a cobalamin-linked diagnostic or therapeutic results in absorption greater than 2 to 5 micrograms per day from the gastrointestinal tract, or greater than one milligram per day when administered via intravenous injection represents a true advance in the art. The teachings of the patents disclosed in the background of the invention do not describe methods to increase the deliverable concentration of a cobalamin-linked diagnostic or therapeutic agent that accomplishes an increase in uptake, bioavailability and/or the diagnostic signal. Contrary to the publication of Seetharam et al., this method can be used in patients that do not exhibit any type of B₁₂ or B₁₂-related deficiency.

The transcobalamin II or intrinsic factor receptor ligand can be a cobalamin, such as vitamin B₁₂, cyanocobalamin, adenosylcobalamin, hydroxycobalamin or methylcobalamin, or a compound of Formula I.

A compound of Formula I can be linked to a diagnostic, therapeutic or other material in combination with an effective amount of a cobalamin transport protein (which term, as used herein, includes but is not limited to intrinsic factor, transcobalamin I, transcobalamin II and transcobalamin III).

The compound of Formula I is of the structure: 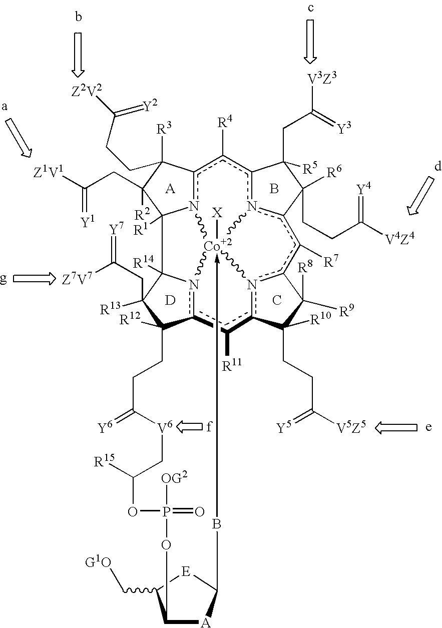
or its enantiomer, diastereomer, salt or prodrug thereof, wherein:
(i) the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;
(ii) the dotted line in the chemical structure indicates either a double or single bond such that the double bond does not over-extend the valence of the element (i.e. to give pentavalent carbons) and, in the case of a single bond, the valence is completed with hydrogen
(iii) X is hydrogen, cyano, amino, amido, hydroxyl, adenosyl L-T, alkyl, alkenyl, alkynyl, cylcoalkyl, aryl, aralkyl, heterocycle, heteroaryl or alkylheteroaryl;
(iv) B is a divalent heterocycle wherein the radical positions can be within the ring or a substituent to the ring such that at least one radical is on a heteroatom to form a dative bond with cobalt, optionally substituted by L-T;
(v) A is O, S, NJ¹, CR¹⁰⁰R¹⁰¹ or C(R¹⁰⁰)V⁸Z⁸;
(vi) E is O or S;
(vii) G¹ and G² are independently hydrogen, alkyl, acyl, silyl, phosphate, or L-T;
(viii) Y¹, Y², Y³, Y⁴, Y⁵, Y⁶ and Y⁷ independently are O, S or NJ²;
(ix) V¹, V², V³, V⁴, V⁵, V⁶, V⁷ and V⁸ independently are O, S or NJ³; CR¹⁰²R¹⁰³, or a direct bond;
(x) Z¹, Z², Z³, Z⁴, Z⁵, Z⁷ and Z⁸ independently are R¹⁰⁴ or L-T;
(xi) each L is independently a direct bond or the residue of a multivalent moiety that does not significantly impair the ability of the compound to bind to a cobalamin transport protein;
(xii) each T is independently a diagnostic or therapeutic agent;
(xiii) at least one of Z¹, Z², Z³, Z⁴, Z⁵, Z⁷, Z⁸, A, B, G¹, and G² comprises an a nucleic acid sequence useful in antisense technology, a peptide nucleic acid or morpholino nucleic acid;
(xiv) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xv) R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(xvi) R¹³ and R¹⁴ optionally can come together to form a pi bond; and
(xvii) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

For diagnosis, either or both of the (i) cobalamin transport protein or the (ii) cobalamin or compound of Formula I linked to a diagnostic, therapeutic or other material, can be labeled with a radioligand or other detectable agent.

For treatment, either or both of the (i) cobalamin transport protein or the (ii) cobalamin or compound of Formula I linked to a diagnostic, therapeutic or other material, can be conjugated to a known therapeutic agent, such as in the case of infection, a known antibiotic; in the case of a cardiovascular disease, a known cardiovascular agent; and in the case of abnormal cellular proliferation, a known anti-proliferative agent or antisense therapeutic.

In various embodiments, the following types of materials can be linked to the cobalamin or compound of Formula I complexes that are coadministered with a cobalamin transport protein, include but are not limited to the following:
(i) a compound useful for the treatment of a disorder associated with abnormal cellular proliferation;
(ii) a compound useful for the treatment of an infectious disease such as an antibiotic or antiviral agent;
(iii) a compound useful in the treatment of a cardiovascular disorder,
(iv) nucleic acids, peptide nucleic acid, morpholino nucleic acid, or other material that affects gene expression, for example, a transcriptional factor,
(v) a compound useful for the radioimaging to image a variety of disease states; and
(vi) a detectable radionuclide or paramagnetic metal atom.

Examples of such conjugated materials are described in detail in the patents or published patent applications cited in the Background of the Invention.

In one embodiment, the invention encompasses a method for increasing the efficiency of delivery of a cobalamin or a compound of Formula I linked to a diagnostic or therapeutic by administering a cobalamin linked diagnostic or therapeutic in combination (covalent, ionic or admixed) with a cobalamin transport protein. The cobalamin transport protein can be intrinsic factor, transcobalamin I, transcobalamin II or transcobalamin III. A cobalamin or a compound of Formula I linked to a diagnostic or therapeutic by administering the cobalamin linked diagnostic or therapeutic in combination with a cobalamin transport protein can be administered via intravenous, parenteral, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, rectal, vaginal or urethral administration including via-suppository, percutaneous, nasal spray, surgical implant, internal surgical paint, infusion pump or catheter.

The cobalamin or compound of Formula I linked diagnostic or therapeutic agent in combination with a cobalamin transport protein can be administered to patients that do not have a cobalamin or cobalamin-related deficiency, such as inherited or acquired cobalamin deficiencies, for example, deficiencies due to the absence of cobalamin transport proteins, such as intrinsic factor, intrinsic factor receptor, or transcobalamin II.

In one embodiment, the cobalamin or a compound of Formula I linked to a diagnostic, therapeutic or other material is orally delivered to a host in combination with intrinsic factor, in a pharmaceutically acceptable carrier. A cobalamin or a compound of Formula I is either administered bound (i.e. either covalently, ionically, datively or via van der Waals attraction), or unbound (i.e. admixed with) to intrinsic factor.

In another embodiment, the cobalamin or the compound of Formula I linked to a diagnostic, therapeutic or other material is administered in combination with transcobalamin I, II or III via intravenous, parenteral, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, rectal, vaginal or urethral administration including via suppository, percutaneous, nasal spray, surgical implant, internal surgical paint, infusion pump or catheter. The cobalamin or the compound of Formula I is either administered bound (i.e. either covalently, ionically, datively or via van der Waals attraction), or unbound (i.e. admixed with) to transcobalamin I, II or III.

In a typical embodiment, the cobalamin or compound of Formula I linked to a diagnostic, therapeutic or other material is administered in any ratio that achieves the desired result. In one embodiment the ratio is one molecule of the cobalamin or compound of Formula I to at least one molecule of cobalamin transport protein. In an alternate embodiment of the invention, the ratio is one molecule of cobalamin or the compound of Formula I to at least one molecule of cobalamin transport protein, and preferably with an excess of cobalamin transport protein, for example, 1.5, 2, 3, 4, 5, or more times excess of cobalamin transport protein. In another embodiment of the invention, the ratio is at least one molecule of the cobalamin or compound of Formula I to one molecule of cobalamin transport protein, and preferably with an excess of the cobalamin or compound of Formula I, for example, 1.5, 2, 3, 4, 5, or more times excess of the cobalamin or compound of Formula I.

The mixtures can be prepared by either physically mixing the cobalamin transport protein with the cobalamin or compound of Formula I linked to a diagnostic, therapeutic or other material prior to formulation in a pharmaceutically acceptable carrier, or by simply mixing them separately with the carrier.

Cobalamin transport proteins, such as IF or transcobalamin I, II or III, can be obtained from any source known in the art. In a particular embodiment, the cobalamin transport protein is extracted from blood by methods known in the art. In an alternate embodiment, the cobalamin transport protein is extracted from cow's milk by methods known in the art.

### DETAILED DESCRIPTION OF THE INVENTION

It has been discovered that when cobalamin derivatives conjugated to therapeutic or diagnostic (i.e., detectable) agents are further conjugated to or administered with a cobalamin transport protein, such as IF or TC-I, -II or -III, more of the active or diagnostic material is absorbed compared administration of the a cobalamin derivative and the therapeutic or diagnostic agent alone.

The invention as disclosed is a method and composition to increase the uptake and bioabsorption of either cobalamin or a compound of Formula I linked to a diagnostic, therapeutic or other material being delivered to a host by administration in combination with an effective amount of a cobalamin transport protein (which term, as used herein, includes but is not limited to intrinsic factor, transcobalamin I, II, and III).

In one embodiment, the cobalamin or compound of Formula I linked to a diagnostic, therapeutic or other material is orally delivered to a host in combination with intrinsic factor, in a pharmaceutically acceptable carrier. A cobalamin or a compound of Formula I is either administered bound (i.e. either covalently, ionically, datively or via van der Waals attraction), or unbound (i.e. admixed with) to intrinsic factor.

In another embodiment, the cobalamin or compound of Formula I linked to a diagnostic, therapeutic or other material is administered in combination with transcobalamin I, II or III via intravenous, parenteral, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, rectal, vaginal or urethral administration including via suppository, percutaneous, nasal spray, surgical implant, internal surgical paint, infusion pump, or via catheter. A cobalamin or a compound of Formula I is either administered bound (i.e. either covalently, ionically, datively or via van der Waals attraction), or unbound (i.e. admixed with) to transcobalamin I, II or III.

In a typical embodiment, the cobalamin or compound of Formula I linked to a diagnostic, therapeutic or other material is administered in any ratio that achieves the desired result. In one embodiment the ratio is one molecule of a cobalamin or a compound of Formula I to at least one molecule of cobalamin transport protein. In an alternate embodiment of the invention, the ratio is one molecule of a cobalamin or a compound of Formula I to at least one molecule of cobalamin transport protein, and preferably with an excess of cobalamin transport protein, for example, 1.5, 2, 3, 4, 5, or more times excess of cobalamin transport protein. In another embodiment of the invention, the ratio is at least one molecule of a cobalamin or a compound of Formula I to one molecule of cobalamin transport protein, and preferably with an excess of a cobalamin or a compound of Formula I, for example, 1.5, 2, 3, 4, 5, or more times excess of a cobalamin or a compound of Formula I.

The mixtures can be prepared by either physically mixing the cobalamin transport protein with a cobalamin or a compound of Formula I linked to a diagnostic, therapeutic or other material prior to formulation in a pharmaceutically acceptable carrier, or by simply mixing them separately with the carrier. Alternatively, the transport protein can be ionically or covalently bound or otherwise conjugated to the cobalamin or compound of Formula I.

For diagnosis, either or both of the (i) cobalamin transport protein or the (ii) cobalamin or compound of Formula I linked to a diagnostic, therapeutic or other material can be labeled with a radioligand or other detectable agent.

As one example, the coadministration of a cobalamin or a compound of Formula I linked to a diagnostic material being delivered to a host allows the detection of solid tumor masses at sizes smaller than previously detectable, because more of the detectable agent is absorbed by the tumor cell. This is especially important for breast cancer patients, because the technology allows the possibility of identifying breast tumor growths at an earlier stage of development, with the possibility of more optimistic prognosis.

I. TC- or IF-Receptor Ligand

One TC- or IF-receptor ligand of the present invention is of the Formula I: 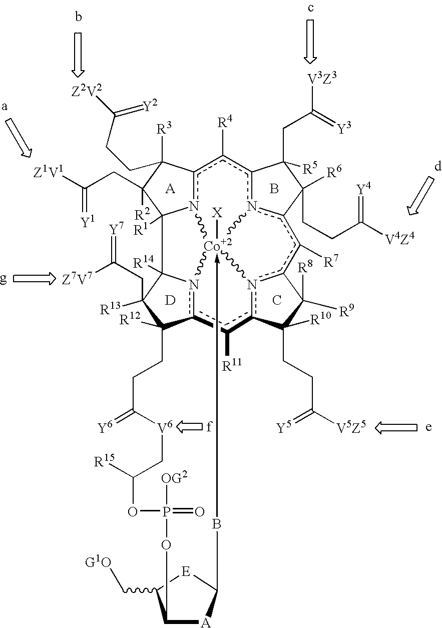
or its enantiomer, diastereomer, salt or prodrug thereof, wherein:
(i) the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;
(ii) the dotted line in the chemical structure indicates either a double or single bond such that the double bond does not over-extend the valence of the element (i.e. to give pentavalent carbons) and, in the case of a single bond, the valence is completed with hydrogen
(iii) X is hydrogen, cyano, amino, amido, hydroxyl, adenosyl L-T, alkyl, alkenyl, alkynyl, cylcoalkyl, aryl, aralkyl, heterocycle, heteroaryl or alkylheteroaryl;
(iv) B is a divalent heterocycle wherein the radical positions can be within the ring or a substituent to the ring such that at least one radical is on a heteroatom to form a dative bond with cobalt, optionally substituted by L-T;
(v) A is O, S, NJ¹, CR¹⁰⁰R¹⁰¹ or C(R¹⁰⁰)V⁸Z⁸;
(vi) E is O or S;
(vii) G¹ and G² are independently hydrogen, alkyl, acyl, silyl, phosphate, or L-T;
(viii) Y¹, Y², Y³, Y⁴, Y⁵, Y⁶ and Y⁷ independently are O, S or NJ²;
(ix) V¹, V², V³, V⁴, V⁵, V⁶, V⁷ and V⁸ independently are O, S or NJ³; CR¹⁰²R¹⁰³, or a direct bond;
(x) Z¹, Z², Z³, Z⁴, Z⁵, Z⁷ and Z⁸ independently are R¹⁰⁴ or L-T;
(xi) each L is independently a direct bond or the residue of a multivalent moiety that does not significantly impair the ability of the compound to bind a cobalamin transport protein;
(xii) each T is independently a diagnostic or therapeutic agent;
(xiii) at least one of Z¹, Z², Z³, Z⁴, Z⁵, Z⁷, Z⁸, A, B, G¹, and G² comprises an a nucleic acid sequence useful in antisense technology, a peptide nucleic acid or morpholino nucleic acid;
(xiv) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xv) R¹, R², R³R R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(xvi) R¹³ and R¹⁴ optionally can come together to form a pi bond; and
(xvii) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

For diagnosis, either or both of the (i) cobalamin transport protein or the (ii) cobalamin or compound of Formula I linked to a diagnostic, therapeutic or other material, can be labeled with a radioligand or other detectable agent.

For treatment, either or both of the (i) cobalamin transport protein or the (ii) cobalamin or compound of Formula I linked to a diagnostic, therapeutic or other material, can be conjugated to a known therapeutic agent, such as in the case of infection, a known antibiotic; in the case of a cardiovascular disease, a known cardiovascular agent; and in the case of abnormal cellular proliferation, a known anti-proliferative agent or antisense therapeutic.

In various embodiments, the following types of materials can be linked to a cobalamin or a compound of Formula I complex that is administered with a cobalamin transport protein, include but are not limited to the following:
(i) a compound useful for the treatment of a disorder associated with abnormal cellular proliferation;
(ii) a compound useful for the treatment of an infectious disease such as an antibiotic or antiviral agent;
(iii) a compound useful in the treatment of a cardiovascular disorder;
(iv) nucleic acids, peptide nucleic acid, morpholino nucleic acid, or other material that affects gene expression, for example, a transcription al factor,
(v) a compound useful for the radioimaging to image a variety of disease states; and
(vi) a detectable radionuclide or paramagnetic metal atom;

Examples of such conjugated materials are described in detail in the patents or published patent applications cited in the Background of the Invention.

In naturally occurring vitamin B₁₂ there is an α-D-5,6-dimethylbenzimidazolyl ribose 3′-phosphate that is bound through the phosphate to the B₁₂ moiety and coordinated to the cobalt ion. In a modified vitamin B₁₂ TC- or IF-receptor ligand, the M-sugar component is typically likewise in an α-D configuration, although other configurations (i.e. α-L, β-D and β-L) are possible.

One of the biologically active forms of vitamin B₁₂ has a 5′-deoxyadenosyl moiety in the X position. Coenzyme B₁₂ catalysis occurs via the detachment and reattachment of the methylene radical at the 5′-deoxy position of the vitamin.

In one particular embodiment the linker used to conjugate the cobalamin or compound of Formula I and the diagnostic or therapeutic agent is a polyamine such as spermine or spermidine.

Because vitamin B₁₂ is preferentially taken up in or near sites of proliferation (either from infection or diseases associated with abnormal cellular proliferation), the cobalamin or compound of Formula I of the present invention provides a delivery system capable of targeting sites of infection or abnormal cellular proliferation and selectively imaging or treating a greater proportion of such sites in relation to healthy cells. A wide range of analogs and derivatives are capable of attaining these properties, as reflected by the above referenced chemical structure and variables.

The cobalamin or compound of Formula I can be modified in any manner that does not interfere with its fundamental ability to bind a cobalamin transport protein, and thereafter bind the TC or IF receptor. In one embodiment, each variable on the vitamin B₁₂ structure independently either (i) retains its natural vitamin B₁₂ structure, (ii) imparts a diagnostic or therapeutic agent to the cobalamin conjugate, (iii) renders the cobalamin conjugate more water soluble or more stable, (iv) increases the bioavailability of the carrier; (v) increases or at least does not decrease the binding affinity of the cobalamin transport protein for the TC-binding or IF-binding protein over vitamin B₁₂; or (vi) imparts another characteristic that is desired for pharmaceutical or diagnostic performance.

The diagnostic or therapeutic agent can be linked to a compound of Formula I through a number of positions, including any of the V-Z moieties, the X moiety, the M moiety, the K moiety and/or the G¹ moiety, though as mentioned above at least one of Z¹, Z², Z³, Z⁴, Z⁵, Z⁷, Z⁸, M and G¹ moieties comprises an diagnostic or therapeutic agent In one embodiment a diagnostic or therapeutic agent is linked to a compound of Formula I through Z², Z⁴, and/or Z⁵ (i.e. one or more of Z², Z⁴ and Z⁵ is L-T and T is a diagnostic or therapeutic agent). In a more particular embodiment a diagnostic or therapeutic agent is linked to a compound of Formula I through the Z² moiety (i.e. Z² is L-T and T a diagnostic or therapeutic agent). In each of the foregoing embodiments, the Z moiety or moieties not containing a diagnostic or therapeutic agent preferably retain its natural vitamin B₁₂ configuration, in which VZ is NH₂. Alternatively, the Z moieties not containing a diagnostic or therapeutic agent may comprise a secondary or tertiary amino analog of NH₂ substituted by one or two of J¹.

In any Z¹, Z², Z³, Z⁴, Z⁵, Z⁶, Z⁷, Z⁸, X, M or G¹ moieties through which a diagnostic or therapeutic agent is linked, it will be understood that such moiety may comprise more than one diagnostic or therapeutic agent, or a combination of agents, i.e. each T can independently comprise the residue of one or more diagnostic or therapeutic agent(s) bound to L through one or more chelating moieties. More specifically, in a series of embodiments, each T can comprise 1, 2, 3, 4, 5, 6, 7, 8, 9 or 10 a diagnostic or therapeutic agent(s) bound through one or more chelating moieties.

R¹, R², R³, R⁴, R⁵, R⁶, R⁷, R⁸, R⁹, R¹⁰, R¹¹, R¹² and R¹³ independently represent moieties that do not interfere with binding between the compound and the cobalamin transport protein or receptor. Vitamin B₁₂ can be modified through these moieties to modulate physical properties of the molecule, such as water solubility, stability or λₘₐₓ. Preferred groups for enhancing water solubility include heteroalkyl, amino, C₁₋₆ alkylamino, C₁₋₆ alcohol, C₁₋₆ carboxylic acid and SO₃⁻.

In another embodiment, one, some or all of R¹, R², R³, R⁴, R⁵, R⁶, R⁷, R⁸, R⁹, R¹⁰, R¹¹, R¹² and R¹³ independently assume their natural roles in vitamin B₁₂. Thus, one, some or all of R¹, R², R⁴, R⁵, R⁸, R⁹, R¹¹, R¹² and R⁵ are independently methyl in one embodiment and one, some or all of R³, R⁶, R⁷, R¹⁰, R¹³ and R¹⁴ are independently hydrogen.

In another embodiment, one, some or all of Y¹, Y², Y³, Y⁴, Y⁵, Y⁶ and Y⁷ assume their natural roles in vitamin B₁₂ and are O. Similarly, in another embodiment V⁶ assumes its natural role in vitamin B₁₂ and is NH or a primary amine analog thereof substituted by J¹.

In still another embodiment, position X assumes its natural role in vitamin B₁₂, i.e. as cyano, hydroxyl, methyl or 5′-deoxyadenosyl, most preferably 5′-deoxyadenosyl.

In another embodiment M is the radical of a purine or pyrimidine base. In another embodiment M is the radical of adenosine, guanine, cytosine, uridine or thymine. In still another embodiment M is the radical of 5,6-dimethylbenzimidazole.

In still another embodiment K is CH(OH).

In yet another embodiment E is O.

In another embodiment G¹ is OH.

In still another embodiment, all constituents of the conjugate assume their natural roles in vitamin B₁₂, except for the moieties through which any diagnostic or therapeutic agent(s) are linked. The diagnostic or therapeutic agent(s) are preferably linked to the vitamin B₁₂ structure through Z², Z⁴ and/or Z⁵ and even more preferably through the Z² moieties.

II. Linkers

As noted above, L is the residue of a linker molecule that conjugates one or more diagnostic or therapeutic agent(s) to a compound of Formula I. The structure of the linker from which L is derived (in any one of the Z¹, Z², Z³, Z⁴, Z⁵, Z⁶, Z⁷, X, M or G¹ moieties) is not crucial, provided it does not significantly impair the ability of the conjugate to bind to the cobalamin transport protein or receptor. L is preferably any multivalent molecule (divalent or greater) that does not significantly impair the ability of the TC carrier to bind to the cobalamin transport protein or receptor. The ability of a cobalamin or a compound of Formula I to bind to the cobalamin transport protein or receptor is “significantly impaired” when attaching a linking moiety to a cobalamin or a compound of Formula I lessens the affinity of the vitamin B₁₂ or the TC-binding carrier for the cobalamin transport protein to which the a cobalamin or a compound of Formula I is most readily bound by 50% or more. The unsaturated vitamin B₁₂ binding capacity (UBBC) assay described by D. A. Collins and H. P. C. Hogenkamp in J. Nuclear Medicine, 1997, 38, 717-723 can be used to compare the relative affinities of ligands for this receptor.

In one embodiment the linker is of precise molecular weight and does not posses a molecular weight distribution. In one embodiment, the linker has a molecular weight less than about 2,500, 2,000, 1900, 1800, 1,500, 1,000 or 500.

A particularly preferred linker is one having multiple sites for conjugation to one or more imaging agents, wherein the linker has a unimodal molecular weight. Recombinant protein production techniques can be employed to obtain poly(amino acid) linkers of substantially constant molecular weight.

In one embodiment the linker is an amino acid or a polymer or peptide formed from a plurality of amino acids. The polymer or peptide can be derived from one or more amino acids. The amino acid, poly(amino acid) or peptide can link T to V through the carboxy terminus or the amino terminus. The amino acid residue, peptide residue or poly(amino acid) residue can conveniently be linked to V and T through an amide (e.g. —N(R)C(—O)— or —C(═O)N(R)—), ester (e.g. —OC(═O)— or —C(═O)O—), ether (e.g. —O—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C₁-C₁₄) alkyl.

Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right, but are meant to also include the opposite flow. Particularly suitable peptides and poly(amino acids) comprise from 2 to about 20 amino acids, from 2 to about 15 amino acids or from 2 to about 12 amino acids.

One exemplary poly(amino acid) is poly-L-lysine ((—NHCH((CH₂)₄—NH₂)CO—)ₘ-Q, wherein Q is H, (C₁-C₁₄)alkyl or a suitable carboxy protecting group and m is from 2 to about 20, from about 5 to about 15 or from about 8 to about 11. The polylysine offers multiple primary amine sites to which active agents can be readily attached. Alternatively, the linkers can be formed with multiple cysteines, to provide free thiols or multiple glutamates or aspartates, to provide free carboxyls for conjugation using suitable carbodiimides. Similarly the linker can contain multiple histidines or tyrosines for conjugation. Other exemplary poly(amino acid) linkers are poly-L-glutamic acid, poly-L-aspartic acid, poly-L-histidine, poly-L-ornithine, poly-L-serine, poly-L-threonine, poly-L-tyrosine, poly-L-lysine-L-phenylalanine or poly-L-lysine-L-tyrosine. When the linker is derived from a poly(amino acid) other than polylysine, the linker is, in a series of embodiments, prepared from 2 to about 30 amino acids, 5 to about 20 amino acids or 8 to about 15 amino acids.

In another particular embodiment L is a polyamine residue (having at least three amino moieties) of the following chemical structure: NR′(alkylene-NR′)ₙalkyleneNR′, wherein n is from 1 to 20, the carbon length of alkylene can vary within the n units and each R′ is independently hydrogen, lower alkyl or T. N is preferably from 1 to 10. Moreover, L preferably has a backbone along its longest length of no more than 100, 75, 50, 40, 30, 20 or 15 atoms. Exemplary polyamines from which L can be derived include spermine (H₂N(CH₂)NH(CH₂)₄NH(CH₂)₃NH₂), spermidine (H₂N(CH₂)₃NH(CH₂)₄NH₂), decamethylene tetraamine and pentamethylene hexamine. These linkers are a definite size and thus provide consistent and predictable targeting by the cobalamin conjugate, in addition to multiple binding sites for the imaging agent.

In another embodiment L is a diamine represented by the formula NH₂ (CH₂)ₓ NH₂, in which x is 2-20 and preferably 2-12. Thus, the linker can be prepared from 1,6-diaminohexane, 1,5-diaminopentane, 1,4-diaminobutane and 1,3-diaminopropane.

Other suitable linkers are formed from the covalent linkage of various water soluble molecules with amino acids, peptides, poly(amino acids), polyamines, polyoxyalkylenes, polyanhydrides, polyesters, polyamides, polyglycolides and diamines. Suitable water soluble molecules include, for example, polyethylene glycol and dicarboxylic monosaccharides such as glucaric acid, galactaric acid and xylaric acid.

Other suitable linkers include those represented by the formula HO(O)C(CH₂)ₓC(O)OH, in which x is 2-20 and preferably 2-12. Thus, the linker can be prepared from succinic acid, glutaric acid, adipic acid, suberic acid, sebacic acid, azelaeic acid or maleic acid. Still other suitable linkers comprise carboxylic acid derivatives that yield an amide upon reaction with an amine. Such reactive groups include, by way of example, carboxylic acid halides such as acid chlorides and bromides; carboxylic acid anhydrides such as acetic anhydrides and trifluoroacetic anhydrides; esters such as p-nitrophenyl esters and N-hydroxysuccinimide esters; and imidazolides. Techniques for using such linkers are described in detail in Bodanszky, Principles of Peptide Synthesis, Springer Verlag, Berlin, 1984.

In one embodiment, the linker is modified to facilitate its conjugation either to V or T. Suitable molecules for modifying the linker include: disuccinimidyl suberate (DSS), bis(sulfosuccinimidyl) suberate (BSS), ethylene glycolbis(succinimidylsuccinate) (EGS), ethylene glycolbis(sulfosuccinimidyl-succinate) (Sulfo-EGS), p-aminophenylacetic acid, dithio-bis-succinimidyl-propionate) (DSP), 3,3′-dithiobis-(sulfosuccinimidylpropionate) (DTSSP), disuccinimidyl tararate (DST), disulfosuccinimidyl tartarate (Sulfo-DST), bis(2-(succinimidooxycarbonyloxy)ethylene)sulfone (BSOCOES), bis(2-(sulfosuccinimidooxy-carbonyloxy)ethylene)sulfone (Sulfo-BSOCOES), dimethyl adipimidate.2HCl (DMA), dimethyl pimelimidate.2HCl (DMP) and dimethyl suberimidate.2HCl (DMS).

Biodegradable Linkers

Various degradable linkers can be used to link a cobalamin or a compound of Formula I to the active agent. The desired linkers can degrade under biological conditions such as by enzymatic cleavage or by systemic pH or temperature. Alternatively, these linkers can be induced to degrade by external manipulation such as changes in pH, temperature, ultrasound, magnetic field, radiation (i.e. UV radiation) or light.

Nonlimiting examples of U.S. Patents that describe controlled release formulations suitable for use as linking agents are: U.S. Pat. No. 5,356,630 to Laurencin et al. (Delivery System for Controlled Release of Bioactive Factors); U.S. Pat. No. 5,797,898 to Santini, Jr. et al. (Microchip Drug Delivery Devices); U.S. Pat. No. 5,874,064 to Edwards et al. (Aerodynamically Light Particles for Pulmonary Drug Delivery); U.S. Pat. No. 5,548,035 to Kim et al. (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al. (Drug Delivery Porphyrin Composition and Methods); U.S. Pat. No. 5,741,329 to Agrawal et al. (Methods of Controlling the pH in the Vicinity of Biodegradable Implants); U.S. Pat. No. 5,820,883 to Tice et al. (Methods for Delivering Bioactive Agents into and Through the Mucosally-Associated Lymphoid Tissues and Controlling Their Release); U.S. Pat. No. 5,955,068 to Gouin et al. (Biodegradable polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et at (Continuous Release Polymeric Implant Carriers); U.S. Pat. No. 6,060,582 to Hubbell et al. (Photopolymerizable Biodegradable Hydrogels as Tissue Contacting Materials and Controlled Release Carriers); U.S. Pat. No. 6,113,943 to Okada et at (Sustained-Release Preparation Capable of Releasing a Physiologically Active Substance); and PCT Publication No. WO 99/59548 to Oh et al (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Improved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No. 5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Implantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 Ionically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-co-anhydride) for biological application; U.S. Pat. No. 4,948,587 Ultrasound enhancement of transbuccal drug delivery, U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, bioerodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

Nonmetallic radioisotopes can conveniently be linked to the vitamin B₁₂ structure through a residue of a peptide having the following formula: 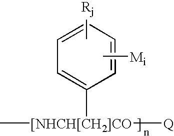
wherein each M is independently a non-metallic radionuclide; each R is independently (C₁-C₁₄)alkyl, (C₂-C₁₄)alkenyl, (C₂-C₁₄)alkynyl, (C₁-C₁₄)alkoxy, hydroxy, cyano, nitro, halo, trifluoromethyl, N(Rₐ)(R_{b}), (C₁-C₁₄)alkanoyl, (C₂-C₁₄)alkanoyloxy, (C₆-C₁₀)aryl or (C₃-C₈)cycloalkyl wherein Rₐ and R_{b} are each independently H or (C₁-C₁₄)alkyl; P; Q is H, (C₁-C₁₄)alkyl or a suitable carboxy protecting group; n is 2 to about 20; I is 1-5, j is 0-4 and I+j is ≦5; or a pharmaceutically acceptable salt thereof. Specifically, i can be 1, j can be 0, M can be a positron emitter such as Fluorine-18, Bromine-76, Iodine-124 or a gamma emitter such as Iodine-123 or Iodine-131 and n can be about 6 to about 12.

The above discussion has demonstrated how the various variables associated with the cobalamin conjugates of the present invention can be independently varied to more particularly define specific classes of cobalamin conjugates encompassed by this invention. It is to be understood that the modification of one variable can be made independently of the modification of any other variable. Moreover, any number of embodiments can be defined by modifying two or more of the variables in such embodiments. A few of such embodiments are described below for purposes of exemplification.

Subembodiment 1: X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, mono-, di- or tri-phosphate or L-T; Y¹, Y², Y³, Y⁴, Y⁵, Y⁶ and Y⁷ independently are O, S or NJ²; V¹, V², V₃, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z¹, Z², Z³, Z⁴, Z⁵, Z⁷ and Z⁸, M or G¹ comprises a diagnostic or therapeutic agent; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 2: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z¹, Z², Z³, Z⁴, Z⁵, Z⁷ and Z⁸, M or G¹ comprises a diagnostic or therapeutic agent; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 3: X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, mono-, di- or tri-phosphate or L-T; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z², Z⁴ or Z⁵ comprises a diagnostic or therapeutic agent, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 4: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹; V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z¹, Z², Z³, Z⁴, Z⁵, Z⁷, Z⁸, M and G¹ comprises a diagnostic or therapeutic agent; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 5: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, LT or L-T; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more radionuclides; at least one of Z², Z⁴ or Z⁵ comprises a diagnostic or therapeutic agent, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 6: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z², Z⁴ or Z⁵ comprises a diagnostic or therapeutic agent, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 7: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z¹, Z², Z³, Z⁴, Z⁵, Z⁷, Z⁸, M and G¹ comprises a diagnostic or therapeutic agent; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 8: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z², Z⁴ or Z⁵ comprises a diagnostic or therapeutic agent, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 9: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z², Z⁴ or Z⁵ comprises a diagnostic or therapeutic agent, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 10: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind cobalamin transport proteins; each L is independently a direct bond or the residue of a multivalent moiety that does not significantly impair the ability of the compound to bind cobalamin transport proteins; each T or T′ independently comprises the residue of one or more diagnostic or therapeutic agent(s); at least one of Z², Z⁴ or Z⁵ comprises a diagnostic or therapeutic agent, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiments 11-20: Any one of subembodiments 1-10, wherein the linker has a substantially constant molecular weight

Subembodiments 21-30: Any one of subembodiments 1-10, wherein the linker is a polyamine of the following chemical structure: NR′(alkylene-NR′)ₙalkyleneNR′, wherein n is from 1 to 20, the carbon length of alkylene can vary within the n units and each R′ is independently hydrogen, lower alkyl or T.

Subembodiments 31-40: Any one of subembodiments 1-10, wherein the linker is spermine, spermidine, decamethylene tetramine or pentamethylene hexamine.

III. Stereoisomerism and Polymorphism

Compounds of the present invention having a chiral center may exist in and be isolated in optically active and racemic forms. Some compounds may exhibit polymorphism. The present invention encompasses racemic, optically-active, polymorphic, or stereoisomeric form, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution.

Additional compounds, intermediates, and synthetic preparations thereof are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691.

IV. Definitions

“Cobalamin transport protein” refers to any of the protein carriers of vitamin B12 or a biologically active metabolite or precursor thereof, including intrinsic factor, transcobalamin I, transcobalamin II or transcobalamin III. “Transcobalamin receptor” or “cobalamin receptor” refers to any receptor to which a cobalamin transport protein conjugate binds.

“Cobalamin” as used herein refers to vitamin B₁₂ or any of its adenosyl, methyl or cyano-derivatives.

Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

The term alkyl, as used herein, unless otherwise specified, refers to a saturated straight, branched, or cyclic, primary, secondary, or tertiary hydrocarbon preferably of C₁ to C₁₀ and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Moieties with which the alkyl group can be substituted are selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term lower alkyl, as used herein, and unless otherwise specified, refers to a C₁ to C₄ saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The terms alkenyl and alkynyl refer to alkyl moieties wherein at least one saturated C—C bond is replaced by a double or triple bond. Thus, (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Similarly, (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “alkylene” refers to a saturated, straight chain, divalent alkyl radical of the formula —(CH₂)ₙ—, wherein n can be 1, 2, 3, 4, 5, 6, 7, 8, 9, or 10.

The term heteroalkyl refers to an alkyl group that contains a heteroatom in the alkyl chain, including O, S, N, or P, and wherein the heteroatom can be attached to other substituents (including R¹⁵) to complete the valence. Nonlimiting examples of heteroalkyl moieties include polyoxyalkylene, and when divalent, —(CH₂O)ₙ— wherein n is an integer of from 0 to 20

The term alkoxy, as used herein, and unless otherwise specified, refers to a moiety of the structure —O-alkyl, wherein alkyl is as defined above.

As used herein, with exceptions as noted, “aryl” is intended to mean any stable monocyclic, bicyclic or tricyclic carbon ring of up to 8 members in each ring, wherein at least one ring is aromatic as defined by the Huckel 4n+2 rule. Examples of aryl ring systems include phenyl, naphthyl, tetrahydronaphthyl and biphenyl. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term heterocycle or heterocyclic, as used herein except where noted represents a stable 5- to 7-membered monocyclic or stable 8- to 11-membered bicyclic heterocyclic ring which is either saturated or unsaturated, and which consists of carbon atoms and from one to three heteroatoms selected from the group consisting of N, O and S; and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom that results in the creation of a stable structure.

The term purine or pyrimidine base includes, but is not limited to, adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-thioalkyl purine, N²-alkylpurines, N²-alkyl-thiopurines, thymine, cytosine, 5-fluorocytosine, 5-methylcytosine, 6-azapyrimidine, including 6-azacytosine, 2- and/or 4-mercapto-pyrmidine, uracil, 5-halouracil, including 5-fluorouracil, C⁵-alkyl-pyrimidines, C⁵-benzylpyrimidines, C⁵-halopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyano-pyrimidine, C⁵-nitropyrimidine, C⁵-aminopyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diamino-purine and 6-chloropurine. Functional oxygen and nitrogen groups on the base can be protected as necessary or desired. Suitable protecting groups are well known to those skilled in the art, and include trimethylsilyl, dimethylhexylsilyl, t-butyldimethylsilyl and t-butyldiphenylsilyl, trityl, alkyl groups, and acyl groups such as acetyl and propionyl, methanesulfonyl, and p-toluenesulfonyl.

The term aralkyl, as used herein, and unless otherwise specified, refers to an aryl group as defined above linked to the molecule through an alkyl group as defined above. The term alkaryl, as used herein, and unless otherwise specified, refers to an alkyl group as defined above linked to the molecule through an aryl group as defined above.

Halo is fluoro, chloro, bromo or iodo.

The term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The term amino, as used herein, refers to a moiety represented by the structure —NR₂, and includes primary amines, and secondary, and tertiary amines substituted by alkyl (i.e. alkylamino). Thus, R₂ may represent two hydrogens, two alkyl moieties, or one hydrogen and one alkyl moiety.

The term amido, as used herein, refers to a moiety represented by the structure —C(O)NR₂, wherein R₂ is as defined for amino.

As used herein, “adenosyl” is an adenosine radical attached to the 6-position of cobalamin via the 5′ position of adenosine.

As used herein, an “amino acid” is a natural amino acid residue (e.g. Ala, Arg, Asn, Asp, Cys, Glu, Gln, Gly, His, Hyl, Hyp, Ile, Leu Lys, Met, Phe, Pro, Ser, Thr, Trp, Tyr, and Val) in D or L form, or an unnatural amino acid (e.g. phosphoserine; phosphothreonine; phosphotyrosine; hydroxyproline; gamma-carboxyglutamate; hippuric acid; octahydroindole-2-carboxylic acid; statine; 1,2,3,4,-tetrahydmisoquinoline-3-carboxylic acid; penicillamine; ornithine; cituline; α-methyl-alanine; para-banzoylphenylalanine; pheylglycine; propargyl-glycine; sarcosine; and tert-butylglycine) residue having one or more open valences. Other unnatural amino acids include those represented by the formula NH₂ (CH₂)_{y} COOH, wherein y=2-20, and preferably 2-12, and include the aminoalkanoic acids such as ε-amino caproic acid (H₂N—(CH₂)₅—COOH).

The term also comprises natural and unnatural amino acids bearing amino protecting groups such as acetyl, acyl, trifluoroacetyl, and benzyloxycarbonyl), as well as natural and unnatural amino acids protected at carboxy with protecting groups such as a C₁-C₆ alkyl, phenyl or benzyl ester and amide. Other suitable amino and carboxy protecting groups are known to those skilled in the art. See for example, T. W. Greene, Protecting Groups in Organic Synthesis; Wiley: New York, 1981; D. Voet, Biochemistry, Wiley: New York, 1990; L. Stryer, Biochemistry, (3^{rd} Ed), W.H. Freeman and Co.: New York, 1975; J. March, Advanced Organic Chemistry. Reactions. Mechanisms and Structure, (2^{nd} Ed.), McGraw Hill: New York, 1977; F. Carey and R. Sundberg, Advanced Organic Chemistry, Part B: Reactions and Synthesis. (2^{nd} Ed.), Plenum: New York, 1977; and references cited therein.

As used herein, a “peptide” is a sequence of 2 to 25 amino acids (e.g. as defined hereinabove) or peptidic residues having one or more open valences. The sequence may be linear or cyclic. For example, a cyclic peptide can be prepared or may result from the formation of disulfide bridges between two cysteine residues in a sequence.

The term host, as used herein, refers to a unicellular or multicellular organism in which the infectious agent can replicate, including cell lines and animals, and preferably a human. Alternatively, the host can be carrying a part of the infectious agent's genome, whose replication or function can be altered by the compounds of the present invention. The term host specifically refers to infected cells, cells transfected with all or part of the infectious agent's genome and animals, in particular, primates (including chimpanzees) and humans. In most animal applications of the present invention, the host is a human patient. Veterinary applications, in certain indications, however, are clearly anticipated by the present invention (such as chimpanzees).

The term “residue” is used throughout the specification to describe any pharmaceutically acceptable form of a diagnostic or therapeutic agent, which, upon administration to a patient, does not inhibit the action of the agent. As a non-limiting example, a pharmaceutically acceptable residue of an agent is one that is modified to facilitate binding to the cobalamin or the compound of Formula I, covalently, ionically or through a chelating agent, such that the modification does not inhibit the biological action of the agent, in that it does not inhibit the drugs ability to modulate the disease. In a preferred embodiment, the residue refers to the agent with an open valence state such that covalent bonding to the compound is possible. This open valence state can be achieved by any means known in the art, including the methodology described herein. In a preferred embodiment, the open valence state is achieved through the removal of an atom, such as hydrogen, to activate a functional group.

The term “pharmaceutically acceptable salt or prodrug” is used throughout the specification to describe any pharmaceutically acceptable form (such as an ester, mono-, di- or tri-phosphate ester, salt of an ester or a related group) of a TC- or IF-binding carrier, which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated; deacylated, phosphorylated, dephosphorylated to produce the active compound. The compounds of this invention possess activity against infectious disease or are metabolized to a compound that exhibits such activity.

V. Diagnostic or Therapeutic Radionuclides

When a detectable radionuclide (e.g., metallic radionuclide) or paramagnetic metal atom is linked to the residue of a compound by a suitable linker, the structure of the linker is not crucial, provided it provides a compound of the invention which has an effective therapeutic and/or diagnostic index against the target cells, and which will localize in or near the disease.

Suitable linkers include linkers that separate the residue of a compound and the detectable radionuclide by about 5 angstroms to about 200 angstroms, inclusive, in length. Other suitable linkers include linkers that separate the residue of a compound of formula I and the detectable radionuclide by about 5 angstroms to about 100 angstroms, as well as linkers that separate the residue of a compound and the detectable radionuclide by about 5 angstroms to about 50 angstroms, or by about 5 angstroms to about 25 angstroms. Suitable linkers are disclosed, for example, in U.S. Pat. No. 5,735,313.

The compounds disclosed herein can be prepared using procedures similar to those described in U.S. Pat. No. 5,739,313, or using procedures similar to those described herein. Additional intermediates and synthetic preparations useful for preparing compounds of the present invention are disclosed, for example, in Hogenkamp, H. et al., Synthesis and Characterization of nido-Carborane Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; and references cited therein.

The compounds disclosed herein can be prepared using procedures similar to those described in U.S. Pat. No. 5,739,313, or using procedures similar to those described herein. The residue of an antibiotic can be linked to the residue of a compound of formula I as described hereinabove. The detectable radionuclide can be linked to the residue of a compound of formula I as described hereinabove. Additional intermediates and synthetic procedures useful for preparing intermediates of the invention are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; U.S. Pat. No. 5,739,313; U.S. Pat. No. 6,004,533; and references cited therein.

In general, the metallic radionuclides (i.e. metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-67, Erbium-169, Europium-152, Gallium-64, Gallium-68, Gadolinium-153, Gadolinium-157 Gold-195, Gold-199, Hafnium-175, Hafnium-175-181, Holmium-166, Indium-110, Indium-111, Iridium-192, Iron-55, Iron-59, Krypton-85, Lead-210, Lutetium-177, Manganese-54, Mercury-197, Mercury-203, Molybdenum-99, Neodymium-147, Neptunium-237, Nickel-63, Niobium-95, Osmium-185+191, Palladium-103, Platinum-195m, Praseodymium-143, Promethium-147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65 and Zirconium-95.

Specifically, the metallic radionuclide can be a diagnostic gamma emitter (e.g., Tc-99m, In-111, Iodine-131, or Iron-59); a diagnostic metallic positron emitting radionuclide (e.g., Bismuth-206, Bismuth-207, Cobalt-55, Gallium-44, Copper-67, Yttrium-86, or Yttrium-88); a paramagnetic diagnosis metal ion (e.g., Europium-152 or Gadolinium-157), or a diagnostic paramagnetic metal ion.

In general, the non-metallic radionuclide is a non-metallic paramagnetic atom (e.g. Fluorine-19); or non-metallic positron emitting radionuclide (e.g. Carbon-11, Fluorine-18, Iodine-12 or Bromine-76) or a nonmetallic gamma emitting radionuclide such as Iodine-123 or Iodine-131. Fluorine-19 is a suitable non-metallic paramagnetic for use the compounds of the present invention in part because there is typically little or no background noise associated with the diagnostic use of fluorine in the body of a mammal (e.g. human).

Compound of Formula I/Linker/Diagnostic Agent—Detectable Radionuclide

As used herein, a “detectable radionuclide” is any suitable radionuclide (i.e. radioisotope) capable of being detected in a diagnostic procedure in vivo or in vitro. Suitable detectable radionuclides include metallic radionuclides (i.e. metallic radioisotopes) and non-metallic radionuclides (i.e. non-metallic radioisotopes).

Compound of Formula I/Linker/Therapeutic Agent—Therapeutic Radionuclide

As used herein, a “therapeutic radionuclide” is any suitable radionuclide (i.e., radioisotope) that possesses therapeutic efficacy against an infectious disease in vivo or in vitro. Suitable therapeutic radionuclides include metallic radionuclides (i.e., metallic radioisotopes).

Specifically, the metallic radionuclide can be a therapeutic metallic radionuclide (e.g., Actinium-223, Bismuth-212, Indium-111, Rhenium-186, Rhenium-188, Strontium-89, Tin-117m, and Yttrium-90) or a therapeutic paramagnetic metal ion (e.g., Gadolinium-157).

VI. Antibiotics as Therapeutic Agents

As used herein, an “antibiotic agent” is any compound having activity against either Gram-positive or Gram-negative organisms (i.e., inhibits the growth or destroys the development of either Gram-positive or Gram-negative organisms) or alternatively a fungus, yeast, or virus. Stedman's Medical Dictionary, Illustrated, (25th Ed.), Williams & Wilkins: Baltimore (1990) and Mosby's Medical, Nursing, & Allied Health Dictionary, (5th Ed.), Mosby: St. Louis (1998).

Infectious diseases include, e.g., acute lower respiratory infections (e.g., pneumonia), lower urinary tract infections (UTI), tuberculosis (TB), Lyme's disease, malaria, meningitis, meningitis caused by Neisseria meningitis, hepatitis, measles, neonatal tetanus, diarrheal diseases (e.g., including cholera, typhoid and dysentery), whooping cough (pertussis), intestinal worm diseases, and sexually transmitted diseases.

Some of the causative agents, and diseases associated with them, include Rotavirus, a major cause of infantile diarrhea worldwide; Cryptosporidium parvum, a parasite which causes acute and chronic diarrhea; Legionella pneumophila, the bacterium which causes potentially fatal Legionnaires' disease; Ebola virus, which causes hemorrhagic fever—fatal in up to 80% of cases; Hantaan virus, which causes potentially fatal hemorrhagic fever with renal syndrome; Campylobacter jejuni, a bacterium which causes diarrhea; Human T-lymphotropic virus I (HTLV-1), which causes lymphoma-leukemia; Escherichia coli O157:H7 strain of bacteria, which causes bloody diarrhea; HTLV-2 virus, which causes hairy cell leukemia; Helicobacter pylori, the bacterium associated with peptic ulcer disease and stomach cancer; Human immunodeficiency virus (HIV), which causes AIDS; Hepatitis E virus, which causes epidemics of jaundice in hot climates; Human herpesvirus 6, which causes fever and rash; Hepatitis C virus, which causes liver cancer as well as liver disease; Guanarito virus, which causes Venezuelan hemorrhagic fever; Vibrio cholerae O139, which causes epidemic cholera; Sabia virus, which causes Brazilian hemorrhagic fever; and Human herpesvirus 8, associated with Kaposi's sarcoma in AIDS patients.

Suitable antibiotic agents are disclosed, e.g., in Physician's Desk Reference (PDR). Medical Economics Company (Montvale, N.J.), (53rd Ed.), 1999; Mavo Medical Center Formulary, Unabridged Version, Mayo Clinic (Rochester, Minn.), January 1998; Merck Index An Encyclopedia of Chemicals, Drugs, and Biologicals, (11th Ed.), Merck & Co., Inc. (Rahway, N.J.), 1989; University of Wisconsin Antimicrobial Use Guide, http://www.medsch.wisc.edu/clinsci/amcg/amcg.html; Introduction on the Use of the Antibiotics Guideline, Descritions of Specific Antibiotic Classes, Thomas Jefferson University, http://jeffline.tju.edu/CWIS/OAC/antibiotics&uide/intro.html; and references cited therein.

Suitable antibiotics include, e.g., amiinoglycosides, β-lactam antibiotics, cephalosporins, macrolides, miscellaneous antibiotics, penicillins, tetracyclines, antiftngals, antimalarial agents, antituberculosis agents, antivirals, leprostatics, miscellaneous anti-infectives, quinolones, sulfonamides, urinary anti-infectives, nasal antibiotics, opthalmic antibiotics, opthalmic antivirals, opthalmic quinalones, opthalmic sulfonamides, skin and mucous membrane antibiotics, skin and mucous membrane antifingals, skin and mucous membrane antivirals, skin and mucous membrane miscellaneous anti-infectives, skin and mucous membrane scabicides and pedulicides, skin and mucous membrane antineoplasts, nitrofurans, and oxazolidinones. Physician's Desk Reference (PDR) Medical Economics Company (Montvale, N.J.), (53rd Ed.), 1999 and Mayo Medical Center Formulary, Unabridged Version, Mayo Clinic (Rochester, Minn.), January 1998.

Aminoglycosides include, e.g., Amikacin (amikacin sulfate); Garamycin (gentamicin sulfate); Nebcin (tobramrycin sulfate); Netromycin (netilmicin sulfate); Streptomycin Sulfate; and TOBI (tobramycin).

β-Lactam antibiotics include, e.g., Azactam (aztreonam); Cefotan (cefotetan); Lorabid (loracarbef; Mefoxin (cefoxitin); Merrem (meropenem); and Primaxin (imipenem and cilastatin for injectable suspension).

Cephalosporins include, e.g., Ancef (cefazolin); Ceclor (cefaclor); Cedax (ceftibuten); Cefizox (ceftizoxime sodium); Cefobid (cefoperazone sodium); Ceftin (cefuroxime axetil); Cefzil (cefprozil); Ceptaz (ceftazidime); Claforan (cefotaxime); Duricef (cefadroxil monohydrate); Fortaz (ceftazidime); Keflex (cephalexin); Keftab (cephalexin HCl); Keflirox (cefuroxime); Kefzol (cefazolin); Mandol (cefamandole nafate); Maxipime (cefepime HCl); Monocid (cefonicid sodium); Omnicef (cefdinir); Rocephin (ceftriaxone); Suprax (cefixime); Tazicef (ceftazidime); Tazidime (ceftazidime); Vantin (cefpodoxime proxetil); and Zinacef (cefuroxime).

Macrolides include, e.g., Biaxin (clarithromycin); Dynabac (dirithromycin); E.E.S. 200 (Erythromycin Ethylsuccinate); E.E.S. 400 (Erythromycin. Ethylsuccinate); Ery-Ped 200 (Erythromycin Ethylsuccinate); EryPed 400 (Erythromycin Ethylsuccinate); Ery-Tab (Erythromycin delayed-release tablets); Erythrocin Stearate (Erythromycin stearate); Ilosone (erythromycin estolate); PCE Dispertab (erythromycin particles in tablets); Pediazole (erythromycin ethylsuccinate and sulfisoxazole acetyl for oral suspension); Tao (troleandomycin); Zithromax (azithromycin); and Erythromycin.

It is appreciated that those skilled in the art understand that the antibiotic useful in the present invention is the biologically active compound present in any of the antibiotic drugs disclosed above. For example, Azactam (aztreonam) is typically available as an injectable solution. The antibiotic agent, however, is (z)-2-[[[(2-amino-4-thiazolyl) [[(2S,-3S)-2-methyl-4-oxo-1-sulfo-3-azetidinyl]carbamoyl]methylene]amino]oxy]-2-methyl propionic acid. Physician's Desk Reference (PDR) Medical Economics Company (Montvale, NJ). (53rd Ed.), pp. 820-823, 1999.

As used herein, a “residue of an antibiotic” is a radical of an antibiotic having one or more open valences. Any synthetically feasible atom or atoms of the antibiotic can be removed to provide the open valence, provided activity against either Gram-positive or Gram-negative organisms is substantially retained when the radical is attached, either directly or via a linker, to a residue of a compound of formula I or provided the compound, upon being linked directly or by a linker to a detectable radionuclide or paramagnetic metal atom, can effectively image the infectious disease. Based on the linkage that is desired, one skilled in the art can select suitably functionalized starting materials that can be derived from an antibiotic using procedures that are known in the art

Compound of Formula I/Linker/Therapeutic Agent—Antibiotic Agent

In addition to being directly linked to the residue of a compound, the residue of an antibiotic can also be linked to the residue of a compound by a suitable linker. The structure of the linker is not crucial, provided the resulting compound of the invention has an effective therapeutic index as an antibiotic drug and preferably can be orally administered. Suitable linkers are disclosed, for example, in U.S. Pat. No. 5,735,313; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; and references cited therein.

VII. Cardiovascular Agents as Therapeutics

The compounds of the invention can optionally be administered in conjunction with one or more known cardiovascular drugs. Suitable cardiovascular drugs are disclosed hereinabove as “cardiovascular agents.”

As used herein, a “cardiovascular disease” is any abnormal condition characterized by the dysfunction of the heart or blood vessels. Some examples of cardiovascular diseases are disclosed, e.g., in Yale University School of Medicine Heat Book, Chapter 23, Cardiovascular Drugs, http://www.info.med.yale.edu/library/heartbk, Apr. 16, 1999; Mosby's Medical, Nursing, & Allied Health Dictionary, (5th Ed.), Mosby, St. Louis, Mo., 1998; and Stedman's Medical Dictionary, (25th Ed.), Williams & Wilkins, Baltimore, Md., 1990.

Cardiovascular diseases include arteriosclerotic heart disease (i.e., arteriosclerosis), angina pectoris, myocardial infarction, vascular diseases (e.g., peripheral vascular disease (PVD) and aneurysms), high blood pressure, hypertension, stroke (e.g., thrombotic stroke, hemorrhagic stroke, and embolic stroke), congestive heart failure, valvular disease, rheumatic heart disease, cardiac arrhythmias (e.g., atrial fibrillation, ventricular tachycardia, atrial arrhythmias, ventricular fibrillation, bradyarrhythmia, and premature ventricular contractions), pericarditis, myocarditis, endocarditis, and cardiomyopathies.

A “cardiovascular agent” is any compound useful to treat one or more abnormal conditions associated with the cardiovascular system. Suitable cardiovascular agents are disclosed, e.g., in Physician's Desk Reference (PDR), Medical Economics Company (Montvale, N.J.), (53rd Ed.), 1999; Mayo Medical Center Formulary, Unabridged Version, Mayo Clinic (Rochester, Minn.), January 1998; Yale University School of Medicine Heart Book: Chapter 23, Cardiovascular Drugs, http://www.info.med.yale.edu/library/heartbk, Apr. 16, 1999; Merck Index, An Encyclopedia of Chemicalsm, Drugs and Biologicals, (11th Ed.), Merck & Co., Inc. (Rahway, N.J.), 1989; and references cited therein.

Suitable cardiovascular agents include blood modifiers, adrenergic blockers (peripheral), adrenergic stimulants (central), alpha/beta adrenergic blockers, angiotensin converting enzyme (ACE) inhibitors, angiotensin II receptor antagonists, anti-arrhythmics (groups I, II, III and IV), miscellaneous anti-arrhythmics, 30 anti-lipemic agents, beta adrenergic blocking agents, calcium channel blockers, diuretics, hypertensive emergency agents, inotropic agents, miscellaneous cardiovascular agents, rauwolfia derivatives, vasodilators and vasopressors.

Suitable blood modifiers include anticoagulants (e.g., Coumadin (crystalline warfarin sodium); Fragmin (dalteparin sodium injection); Heparin Lock (heparin lock flush solution); Heparin sodium (heparin sodium); Lovenox (enoxaparin sodium); Norniflo (ardeparin sodium); Orgaran (danaparoid sodium)); antiplatelet agents (e.g., Aggrastat (tirofiban hydrochloride monohydrate); Agrylin (anagrelide hydrochloride); Ecotrin (enteric-coated aspirin); Flolan (epoprostenol sodium); Halfprin (enteric-coated aspirin); Integrilin (eptifibatide); Persantine (dipyridamole); Plavix (clopidogrel bisulfate); ReoPro (abciximab); and Ticlild (ticlopidine hydrochloride)); colony stimulating factors (e.g., Granulocyte colony-stimulating factor (G-CSF) such as Neupogen (filgrastim); Granulocyte-Macrophage colony-stimulating factor (GM-CSF), such as Leukine (sagramostim)); and hematinics (e.g., Anabolic steroids, such as Anadrol-50 (oxymetholone); and Nascobal (cyanocobalamin); and Trinsicon (hematinic concentrate with intrinsic factor); and Erythropoietin, such as Epogen (epoetin alfa); and Procrit (epoetin alfa).

Suitable adrenergic blockers (peripheral) include Cardura (doxazosin mesylate); Dibenzyline (phenoxybenzamine); Hylorel (guanadrel sulfate); Hytrin (terazosin hydrochloride); Minipress (prazosin hydrochloride); and Minizide (prazosin hydrochloridelpolythiazide).

Suitable adrenergic stimulants (central) include Aldoclor (methyldopa and chlorothiazide sodium); Aldomet (methyldopa); Aldomet ester HCL (methyldopate HCl); Aldoril, (methyldopa and hydrochlorothiazide); Catapres (clonidine HCl); Catapres-TTS (clonidine); Clorpres (clonidine hydrochloride and 25 chlortbalidone); Combipres (clonidinehydrochloride and chlorthalidone); and Tenex (guanfacine).

Suitable alpha/beta adrenergic blockers include Coreg (carvedilol); Normodyne (Labetalol); and Trandate (Labetalol).

Suitable angiotensin converting enzyme (ACE) inhibitors include 30 Accupril (quinapril hydrochloride); Altace (ramipril); Captopril; Lotensin (benazepril hydrochloride); Mavik (trandolapril); Monopril (fosinopril sodium tablets); Prinivil (Lisinopril); Univasc (moexipril hydrochloride); Vasotec (enalapril maleate); and Zestril (lisinopril).

Suitable angiotensin II receptor antagonists include Atacand (candesartan cilexetil); Avapro (irbesartan); Cozaar (losartan potassium); and Diovan (Valsartan) HCT™ (Hydrochlorothiazide).

Suitable anti-arrhythmics, group I, include Cardioquin (quinidine polygalacturonate); Ethmozine (moricizine hydrochloride); Mexitil (mexiletine hydrochloride); Norpace (disopyramide phosphate); Norpace CR (controlled release disopyramide phosphate); Procanbid (procainamide hydrochloride extended-release tablets); Quinaglute (Quinidine); Quinidex (quinidine sulfate); Rythmol (propafenone hydrochloride); Tambocor (flecainide acetate); and Tonocard (tocainide HCL). Suitable anti-arrhythmics, group II, include Betapace (sotalol HCL); Brevibloc (esmolol hydrochloride); Inderal (Popranolol); and Sectral (acebutolol).

Suitable anti-arrhythmics, group III include Betapace (sotalol HCL); Cordarone (arniodarone); Corvert (ibutilide fumarate injection); and Pacerone (Amiodarone hydrochloride).

Suitable anti-arrhythmics, group IV, include Calan (verapamil); and Cardizem (diltiazem HCL).

Suitable miscellaneous anti-arrhythmics include Adenocard (adenosine); Lanoxicaps (digoxin); and Lanoxin (digoxin).

Suitable anti-lipemic agents include bile acid sequestrants (e.g., Colestid (microionized colestipol hydrochloride); LoCholest (cholestyramine); and Questran (cholestyramine)); fibric acid derivatives (e.g., Atromid-S (clofibrate); Lopid (gemfibrozil); and TriCor (fenofibrate capsules)); HMG-CoA reductase inhibitors (e.g., Baycol (cerivastatin sodium tablets); Lescol (fluvastatin sodium); Lipitor (atorvastatin calcium); Mevacor (lovastatin); Pravachol (pravastatin sodium); and Zocor (simvastatin)); and Nicotinic Acid (e.g., Niaspan).

Suitable beta adrenergic blocking agents include Betapace (sotalol HCl); Blocadren (Timolol Maleate); Brevibloc (esmolol hydrochloride); Cartrol (carteolol hydrochloride); Inderal (propranolol hydrochloride); Kerlone (betaxolol hydrochloride); Levatol (Penbutolol sulfate); Lopressor (metropolol tartrate); Sectral (acebutolol hydrochloride); Tenormin (atenolol); Toprol-XL (metoprolol succinate, extended release); and Zebeta (bisoprolol furmurate).

Suitable calcium channel blockers include Adalat (nifedipine); Adalat CC (nifedipine); Calan (verapamil hydrochloride); Calan SR (verapamil hydrochloride); Cardene (nicardipine hydrochloride); Cardizem CD (diltiazem hydrochloride); Cardizem (diltiazem hydrochloride); Cardizem SR (diltiazem hydrochloride); Covera-HS (verapamil hydrochloride); Dilator XR (dilitiazem); DynaCirc (isradipine); DynaCirc CR (isradipine); Isoptin SR (verapamil hydrochloride); Nimotop (nimodipine); Norvasc (amlodipine besylate); Plendil (felodipine); Procardia (nifedipine); Procardia XL (nifedipine, extended release); Sular (nisoldipine); Tiazac (diltiazem hydrochloride); Vascor (bepridil hydrochloride); and Verelan (Vempamil Hydrochloride).

Suitable diuretics include carbonic anhydrase inhibitors (e.g., Daranide (dichlorphenamide)); loop diuretics (e.g., Demadex (torsemide); Edecrin (ethacrynic acid); Edecrini sodium (ethacrynic acid); and Lasix (fuirosemide)); potassium-sparing diuretics (e.g., Aldactone (Spironolactone); Dyrenium (triamterene); and Midamor (amiloride)); thiazides and related diuretics (e.g., Diucardin (hydroflumethazide); Diuril (chlorothiazide); Diuril sodiumn (chlorothiazide); Enduron (methyclothiazide); HydroDIURIL (hydrochlorothiazide (HCTZ)); Microzide (hydrochlorothiazide); Mykrox (metolazone); Renese (polythiazide); Thalitone (chlorthalidone USP); and Zaroxolyn (metolazone)).

Suitable hypertensive emergency agents include Hyperstat (diazoxide).

Suitable inotropic agents include Dobutrex (dobutamine hydrochloride); Lanoxicaps (digoxin); and Lanoxin (digoxin); and Primacor (milrinone lactate injection).

Suitable miscellaneous cardiovascular agents include Demser (metyrosine); Inversine (Mecamylamine HCL); Regitine (phentolamine mesylate); and ReoPro (abeiximab).

Suitable rauwolfia derivatives include Diupres (reserpine and chlorothiazide); and Hydropres (reserpine and hydrochlorothiazide).

Suitable vasodilators include coronary vasodilators (e.g., Deponit (Transdermal Nitroglycerin); Dilatrate-SR (isosorbide dinitrate sustained release); Imdur (isosorbide mononitrate); Ismo (isosorbide mononitrate); Isordil (isosorbide dinitrate); Monoket (isosorbide mononitrate); Nitro-Bid (nitroglycerin); Nitro-Dur (nitroglycerin); Nitrolingual (Nitroglycerin in propellants, Dichlorodifluoromethane and Dichlorotetrafluoromethane); Nitrostat (nitroglycerin); Sorbitrate (isosorbide dinitrate); and Transderm-Nitro (nitroglycerin)); peripheral vasodilators (e.g., Corlopam (fenoldopam mesylate); Flolan (epoprostenol sodium); and Primacor (milinone lactate injection)).

Suitable vasopressors include Ana-Kit (epinephrine); Aramine (etaraminol bitartrate); EpiPen (epinephrine); ProAmatine (midodrine hydrochloride); and Vasoxyl (methoxamine hydrochloride).

It is appreciated that those skilled in the art understand that the cardiovascular agent useful in the present invention is the biologically active compound present in any of the cardiovascular compositions disclosed above. For example, Cardizem (diltiazem HCL) is typically available as an injectable, as a sustained release capsule and as a direct compression tablet. The cardiovascular agent, however, is (+)cis-1,5-benzothiazepin-4 (5H)one,3-(acetyloxy)-5-[2-(dimethyl-amino)ethyl]-2,3-dihydro-2-(4-methoxyphenyl)-monohydro-chloride. Physician's Desk Reference (PDR), Medical Economics Company (Montvale, N.J.), (53rd Ed.), pp. 1311-1318, 1999.

Compound of Formula I/Linker/Therareutic Apent—Cardiovascular Agent

In addition to being directly linked to the residue of a compound, the residue of a cardiovascular agent can also be linked to the residue of a compound by a suitable linker. The structure of the linker is not crucial, provided the resulting compound of the invention has an effective therapeutic index as a cardiovascular drug and preferably can be orally administered. Suitable linkers are disclosed, for example, in U.S. Pat. No. 5,735,313; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; and references cited therein.

VIII. Antiproliferative Agents as Therapeutics

Proliferative disorders are currently treated by a variety of classes of compounds including alkylating agents, antimetabolites, natural products, enzymes, biological response modifiers, miscellaneous agents, hormones and antagonists, such as those listed below.

Alkylating Agents include (1) nitrogen mustards: Mechlorethamine, Cyclophosphamide Ifosfamide, Melphalan (L-sarcolysin), Chlorambucil; (2) Ethylenimines and Methylmelamines: Hexamethylmelamine, Thiotepa; (3) Alkyl Sulfonates: Busulfan, (4) Nitrosoureas: Carmustine (BCNU), Lomustine (CCNU), Semustine (methyl-CCNU), Streptozocin (streptozocin); and (5) Triazenes: Dacarbazine (DTIC; dimethyltriazenoimid-azolecarboxamide).

Antimetabolites include (1) Folic Acid Analogs: Methotrexate (amethopterin); (2) Pyrimidine Analogs: Fluorouracil (5-fluorouracil; 5-FU) Floxuridine (fluorodeoxyuridine; FUDR), Cytarabine (cytosine arabinoside); (3) Purine Analogs: Mercaptopurine (6-mercaptopurine; 6-MP), Thioguanine (6-thioguanine: TG), Pentostatin (2′-deoxycyoformycin); (4) Vinca Alkaloids: Vinblastine (VLB), Vincristine; and (5) Epipodophyl-lotoxins: Etoposide, Teniposide.

Hormones and Antagonists include (1) Estrogens: Diethylstibestrol Ethinyl estrdiol; (2) Antiestrogen: Tamoxifen; (3) Androgens: Testosterone propionate Fluxomyesterone; (4) Antiandrogen: Flutamide; and (5) Gonadotropin-Releasing Hormone Analog: Leuprolide.

Other miscellaneous agents useful in the treatment of abnormal cellular proliferation include (1) Antibiotics: Dactinomycin (actinonmycin D), Daunorubicin (daunomycin; rubidomycin), Doxorubicin, Bleomycin, Plicamycin (mitlrunycin), Mitomycin (mitomycin C); (2) Enzymes: L-Asparaginase; (3) Biological Response Modifiers: Interferon-α; (4) Platinum Coordination Complexes: Cisplatin (cis-DDP), Carboplatin; (5) Anthracenedione: Mixtozantrone; (6) Substituted Urea: Hydroxyurea; (7) Methylhydrzinhe Derivative: Procarbazine (N-methylhydrazine, MbI); (8) Adrenocortical Suppressant: Miotane (o,p′-DDD), Aminoglutethinide; (9) Adrenorticosteriods: Prednisone; and (10) Progestins: Hydroxprogesterone caproate, Medroxyprogersterone acetate, Megestrol acetate.

It has been discovered that a neutron capture agent such as a molecule comprising Boron-10, for the treatment of a proliferative disorder, is highly and effectively absorbed into a site of unwanted proliferation by direct or indirect attachment to a compound that binds to a cobalamin transport protein for vitamin B₁₂ (i.e. transcobalamin I, II or III, or intrinsic factor) (the TC- or IF-binding carrier) in a manner that allows binding to a transcobalamin receptor (TR). Subsequent initiation of neutron capture therapy will selectively destroy abnormally proliferating cells.

It is preferred that the cobalamin or compound of Formula I and the neutron capture agent be administered parenterally, not orally, to increase bioavailability and delivery to proliferative tissue. Importantly, it has been discovered that oral administration of the cobalamin or compound of Formula I/neutron capture agent provides insufficient bioavailability to treat proliferative disorders. It is important, and perhaps essential, to administer the neutron capture agent in a manner that does not rely on the ileal intrinsic factor receptor binding absorption pathway of the active agent.

Compound of Formula I/Linker/Therapeutic Agent—Antiproliferative Agent

In addition to being directly linked to the residue of a compound, the residue of an antiproliferative agent can also be linked to the residue of a compound by a suitable linker. The structure of the linker is not crucial, provided the resulting compound of the invention has an effective therapeutic index as an antiproliferative drug and preferably can be orally administered. Suitable linkers are disclosed, for example, in U.S. Pat. No. 5,735,313; U.S. Application Ser. No. 60/129,733 filed 16 Apr. 1999; U.S. Application Ser. No. 60/159,874 filed 15 Oct. 1999; U.S. Application Ser. No. 60/159,753 filed 15 Oct. 1999; U.S. Application Ser. No. 60/159,873 filed 15 Oct. 1999; and references cited therein.

Thus, in one embodiment the invention provides a neutron capture conjugate having a high specificity for abnormally proliferative cells, comprising (1) a cobalamin or a compound of Formula I, (2) a neutron capture agent linked directly or through a linker to the cobalamin or compound of Formula I, wherein the linker has either (i) a unimodal (i.e., single) and defined molecular weight, or (ii) a molecular weight less than about 2000, and preferably, below 1900, 1800 or 1500; and (3) a cobalamin transport protein (such as IF or TC-I, II or Im).

IX. Antisense Oligonucleotides as Therapeutic Agents

The present invention can be utilized to deliver polynucleic acids, to various kinds of organisms, preferably mammals, more preferably humans, in need thereof by suitably selecting a polynucleic acid sequence in compliance with its use and conjugating the polynucleic acid sequence to a ligand for the transcobalamin receptor or a ligand for the intrinsic factor-cobalamin receptor. The polynucleic acids can be conjugated to a complex of cobalamin transport protein bound to a cobalamin or a compound of Formula I. The present invention can be used to treat diseases by delivering to cells expressing transcobalamin receptors or IF receptors nucleic acid sequences that regulate the expression of specific genes or encode for specific proteins or fragments of proteins.

The polynucleic acid can be any antisense oligonucleotide (optionally a stabilized oligonucleotide), PNA or MNA of short (less than 20 nucleotides), intermediate (between 20 and 100 nucleotides) or long chain length (greater than 100 nucleotides), as desired, doubly or singly stranded. In a preferred embodiment the polynucleic acid sequence can be an antisense RNA, an antisense oligonucleotide, antisense PNA or antisense MNA of 20 nucleotides or less.

In particular, the antisense nucleotides that can be conjugated to the carriers of the present invention are distinguished in Table 1.

TABLE 1

| Name and | | | Status |
|---|---|---|---|
| Sponsor | Sequence | Target/Disease | (Phase) |
| Fomivirsen | GCGTTTGCTCTTCTT CTTGCG | IE-2/CMV Retinitis | FDA |
| (Isis) | | | |
| 2302 (Isis) | GCCCAAGCTGGCATCCGTCA | 3′-UTR/ICAM-1, | II A/B |
| | | Crohn's Disease, | |
| | | Psoriasis, | |
| | | Rheumatoid | |
| | | Arthritis, Ulcerative | |
| | | Colitis, Renal | |
| | | Allograft | |
| 3521/CPG, | GTTCTCGCTGGTGAGTTTCA | 3′-UTR/PKC-a, | II A |
| 64128A (Isis/ | | Ovarian Cancer | |
| Novartis) | | | |
| 5132/CPG, | TCCCGCCTGTGACATGCATT | c.RAF kinase, | I/II |
| 69846A (Isis/ | | Breast, prostrate, | |
| Novartis) | | colon, brain, | |
| | | ovarian cancer | |
| 2503 (Isis) | TCCGTCATCGCTCCTCAGGG | Ha-ras oncogene | I |
| | | variety of solid | |
| | | tumors | |
| G3139 | TCTCCCAGCGTGCGCCAT | bcl-2, Proto- | I/lI A |
| (Genta) | | oncogene, Non- | |
| | | Hodgkin's, | |
| | | Lymphoma, | |
| | | Prostrate, Breast | |
| LR3280 | AACGTTGAGGGCAT | c-myc/proto- | I |
| (Lynx) | | oncogene, Stent | |
| | | Restenosis | |
| LR3001 | TATGCTGTGCCGGGG | c-myb, Proto- | II |
| (Lynx) | TCTTCGGGC | oncogene, Chronic | |
| | | Myeloid, Leukemia | |
| LR4437 | GGACCCTCCTCCGGA GCC | IGF-IR, Ex-vitro | I |
| (Lynx) | | tumor cells | |
| GEM-132 | UGGGGCTTACCTTGC GAACA | Intron-exon, | I/II |
| (Hybridon) | | UL36/27, CMV- | |
| | | retinitis | |
| GEM-92 | UCGCACCCATCTCTC TCCUUC | Gag/HIV-1, AIDS | I |
| (Hybridon) | | | |
| GEM-231 | GCGUGCCTCCTCACUGGC | pka-1, Refractory | I |
| (Hybridon) | | Solid Tumors | |
| GPI-2A | G(ps)GTTC(ps)TTTTG(ps)G(ps)TCC(p | Gag/HIV-1, AIDS | I |
| (Novophann) | s)TTG(ps)TC(ps)T | | |
| 13312 (Isis) | GC(ps)GTTTGC(ps)TC(ps)TTC(ps)TT | IE-2, CMV retinitis | I |
| | C(ps)TTGCG | | |

Note:
The underlined bases in GEM-132, GEM-92, and GEM-231 are 2′OMe sugar modifications.
In GPI-2A, there are seven PS linkages represented by (ps) and the rest of the oligo is a phosphodiester.
In 13312, the underlined bases are 2′-O(CH₂)₂OCH₃ sugar modifications and all U and C residues are 5-methyl substituted.
Cited from: Sanghvi, Y. S. et al. in Manuals of Antisense Methodology.Eds., Hartmann, G., and Endres, S., Kiuwer Academic Publisher, 1998, In Press.

X. Cobalamin Transport Proteins

In humans, the average daily intake (in a Western diet) of vitamin B₁₂ is about 4-5 μg. Additional synthesis of cobalamin may be produced in the ileum and the right colon, but in an unknown amount. The total lumenal cobalamin that must be assimilated each day in humans is estimated at 7-14 μg, the sum of the dietary and endogenous cobalamin. Intestinal epithelial cells possess carriers and transporters that are highly efficient in the uptake of the small products of digestion, such as vitamins, minerals and amino acids. These mechanisms are necessary for the uptake of these molecules, as the epithelial cell layer presents an almost impenetrable barrier to peptides larger than five or six amino acids in size. The cobalamins of the present invention are large molecules that are not absorbed directly from the intestine, as they are too big to diffuse across the intestinal wall. Therefore, the absorption of the cobalamins is dependent upon transport proteins. The uptake of vitamin B12 from the intestine to the blood is perhaps the most complex uptake mechanism of all the vitamins, involving at least four separate cobalamin binding proteins and receptors.

Three distinct groups of transport proteins are involved in the absorption and transport of cobalamins: intrinsic factor (IF), haptocorrin (HC; also called R-protein; in which transcobalamin I (TC-I) and trarscobalamin III (TC-III) are members) and transcobalamin II (TC-II). Both IF and TC II deficiencies lead to abnormalities such as megaloblastic anemia and demyelinating disorder of the nervous system. Each protein only has one subunit and one binding site to cobalamin. IF is a 45 kDa (in humans) to 55 kDa (in hogs) plasma glycoprotein with 15% carbohydrate content. HC's are 58 kDa (in humans) to 60 kDa (in rabbits) plasma glycoproteins of 33-40% carbohydrate content with 16-19 sialic acid residues. Human TC-II is a 43 kDa plasma protein (in humans) with 0% carbohydrate content. Each binding protein has a separate affinity for cobalamin, as well as separate cell receptors. Generally, cobalamin is initially bound by HC in the stomach, followed by IF in the small intestine. An IF receptor is then involved in the uptake of the IF-cobalamin complex by the intestinal epithelial cell, leading to the proteolytic release of cobalamin, and subsequent binding to TC-II.

Intrinsic factor (IF) and haptocorrin (HC; are the main intestinal lumenal cobalamin binders. In particular IF is of particular relevance to the field of oral peptide and protein delivery. Therefore, IF is mainly produced in the gastric body and medium sized ducts and HC is mainly produced in granulocytes, the yolk sac, mammary glands, salivary acini and ducts. In general, in plasma or serum, cobalamin is also bound to HC (derived from white cells) or to TC-II. The former complex is taken up by the liver, delivering flee cobalamin to the intestinal lumen as the first limb of an enterohepatic circulation.

IF is the most specific of the cobalamin-binding proteins. Cyanocobalamin, hydoxy-cobalamin (HOCbl), methylcobalaamin (MeCbl) and adenyosylcobalamin (AdoCbl) bind to intrinsic factor with similar affinities, thereby suggesting that the upper β-axial ligand of the cobalt does not influence the binding significantly. However, after dietary release of vitamin B₁₂, the affinity for the cobalamin for IF is reduced, due to the low pH. Rather, the released vitamin B₁₂ is preferentially bound to salivary HC, as HC may protect the vitamin from acid hydrolysis (possibly due to the extensive glycosylation of HC).

HC comprises a group of immunologically identical proteins secreted into many body fluids (plasma, milk, amniotic fluid, saliva and gastric juices) from many types of cells (granulocytes, mammary glands, yolk sac or visceral placental membranes, salivary duct and acinar cells, and gastric mucosa of some species). These proteins were known previously as R proteins (for rapid electrophoresis), non-intrinsic factors or transcobalamin I and III. They are characterized by different glycosylation processes and account for much of the total bound cobalamin in the serum (about 80% of bound cobalamin in serum). HC turns over very slowly (t_{1/2}=10 days) and appears to serve as the major storage protein for cobalamin and may also stabilize serum cobalamin against transdermal photolysis (Allen, R. H. Prog Hematol. 1975, 9, 57-84).

Within the proximal small intestine, HC is degraded by pancreatic enzymes, freeing cobalamin to combine with other transport proteins, most notably IF. In contrast to HC's, the IF-cobalamin complex is resistant to proteolytic digestion. Once the cobalamin-transport protein is internalized via receptor-mediated endocytosis, the cobalamin is cleaved from transport protein via protease(s) and bound to transcobalamin II (TC II). From there, the TC II-cobalamin complex is used for the transport of absorbed cobalamin to peripheral tissues. Therefore, TC-II is found in most tissues. Antibodies to TC II inhibit the transport of cobalamins and block the proliferation of leukemic cells in vitro (McLean, G. R. et al. Blood. 1997, 89, 235-242). In cow's milk, in particular, the major cobalamin binder is not HC, but rather TC-II (Fedosov, S. N. et al. Biochemistry 1995, 34, 16082-16087 and Fedosov, S. N. et al. Biochim. Biophvs. Acta. 1996, 1292, 113-119).

Early attempts to purify transport proteins utilized conventional techniques such as ammonium sulfate fractionation, ion exchange and size exclusion chromatography. These methods yielded a product that was devoid of the other types transport proteins, and in particular, separation of TC-II from TC-I was possible, but contained other plasma proteins. The introduction of affinity chromatography provided pure proteins even in extremely low concentrations. Three main types of affinity columns have been used to purify the transport proteins, in particular, columns containing cobalamin coupled to different matrices. The first was a monocarboxylic acid derivative of cobalamin linked to Sepharose beads via a diamino-dipropylamine spacer arm (Allen, R. H. et al. J. Biol. Chem. 1972, 247, 7695-7701 and Allen, R. H. et al. J. Biol. Chem. 1973, 248, 3660-3669). However, it may be necessary to use a chaotropic reagent to elute the protein from the matrix, possibly resulting in a denatured transport protein, which may not be able to renature. For instance, the elution of the bound protein from Cohn fraction III of human plasma, a mixture that contains 27-40% of the plasma TC-II, required the use of guanidine hydrochloride to release the denatured TC-II, which could not be renatured.

To avoid the use of chaotropic reagents, temperature- or photolabile linkages between the cobalamin and the insoluble matrix were developed (Nexo, E. “Cobalamin binding proteins,” inVitamin B₁₂ and B₁₂-proteins, eds Krantler, B.; Arigoni, D. and Golding, B. T.; Wiley & Sons, Ltd. 461-475). Matrices formed in this manner are able to release the transport protein by dissociating the cobalamin from the matrix, thus providing the transport protein saturated with cobalamin, circumventing the denaturant effect of chaotrophic agents.

In a preferred embodiment, for large scale purification of transport protein, ion exchange chromatography or ammonium sulfate fractionation is used prior to the purification of the transport protein via an affinity column to concentrate the sample. In an alternate embodiment, ion exchange or size exclusion chromatography is used subsequent to the purification of the transport protein via an affinity column.

XI. Pharmaceutical Dosage Forms

The mode of administration of the cobalamin or compound of Formula I conjugated to a diagnostic or therapeutic agent, bound to a cobalamin transport protein such as intrinsic factor or transcobalamin I, II or III will depend upon the location and nature of the disease, as known to workers skilled in the art. The cobalamin or compound of Formula I conjugated to a diagnostic or therapeutic agent, bound to a cobalamin transport protein such as intrinsic factor or transcobalamin I, II or III can be formulated as pharmaceutical compositions and administered to a mammalian host such as a human patient in a variety of forms adapted to the chosen route of administration, i.e., orally or parenterally, by intravenously, intramuscularly, or subcutaneously, sublingually, mucosally (e.g. nasally), inhalation, transdermally, intra-articular, intra-synovial, intrathecal, intra-artedal, intracardiac, intraorbital, intracapsular, ophthalmically, intraspinal, intrasternal, topical, transdermal patch, via rectal, vaginal or urethral suppository, peritoneal, percutaneous, surgical implant, internal surgical paint, infusion pump or catheter. For standard information on pharmaceutical formulations, see Ansel, et al., Pharmaceutical Dosage Forms and Drug Delivery Systems, Sixth Edition, Williams & Wilkins (1995).

The cobalamin or compound of Formula I/diagnostic or therapeutic agents/cobalamin transport protein can, for example, be administered intravenously or intraperitoneally by infusion or injection. Solutions of the substance can be prepared in water, optionally mixed with a nontoxic surfactant. Dispersions can also be prepared in glycerol, liquid polyethylene glycols, triacetin and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the substance which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, normal saline, ethanol, a polyol (for example, glycerol, propylene glycol, liquid polyethylene glycols and the like), vegetable oils, nontoxic glyceryl esters and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, benzyl alcohol, sorbic acid, thimerosal and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the substance in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Injectable solutions are particularly advantageous for local administration of the therapeutic composition. In particular, parenchymal injection can be used to deliver the therapeutic composition directly to a tumorous growth. Intra-articular injection is a preferred alternative in cases of arthritis where the practitioner wishes to treat one or only a few (such as 2-6) joints. Additionally, the therapeutic compounds are injected directly into lesions (intra-lesion administration) in appropriate cases. Intradermal administration is an alternative for dermal lesions.

The therapeutic compound is optionally administered topically by the use of a transdermal therapeutic system (see, Barry, Dermatological Formulations, (1983) p. 181 and literature cited therein). Transdermal drug delivery (TDD) has several advantages over oral delivery. When compared to oral delivery, TDD avoids gastrointestinal drug metabolism, reduces first pass effects and provides a sustained release of drugs for up to seven days (Elias, et al. Percutaneous Absorption: Mechanisms-Methodology-Drug Delivery; Marcel Dekker, NY: 1, 1989). This method is especially useful with many therapeutic proteins that are susceptible to gastrointestinal degradation and exhibit poor gastrointestinal uptake. When compared to injections, TDD eliminates the associate pain and the possibility of infection. While such topical delivery systems have been designed largely for transdermal administration of low molecular weight drugs, by definition they are capable of percutaneous delivery. They can be readily adapted to administration of the therapeutic compounds of the invention by appropriate selection of the rate-controlling microporous membrane. Topical application can also be achieved by applying the compound of interest, in a cream, lotion, ointment or oil based carrier, directly to the skin. Typically, the concentration of therapeutic compound in a cream, lotion or oil is 1-2%.

For drug targeting to lung tissue, the therapeutic compound is formulated into a solution, suspension, aerosol or particulate dispersion appropriate for application to the pulmonary system. The therapeutic agent may be inhaled via nebulizer, inhalation capsule, inhalation aerosol, nasal solution, intratracheal as a solution via syringe or endotracheal tube as an aerosol or via as a nebulizer solution. Aerosols are prepared using an aqueous aerosol, liposomal preparation or solid particles containing the compound. A nonaqueous (e.g. fluorocarbon propellant) suspension could be used. Sonic nebulizers are preferred because they minimize exposing the therapeutic compound to shear, which can result in degradation of the compound.

Delivery of the cobalamin conjugates of the instant invention by the mucosal route also offers an attractive administration alternative. The prototype formulation for nasal solutions will contain the cobalamin or compound of Formula I conjugate dissolved in a suitable aqueous or non-aqueous solvent such as propylene glycol, an antioxidant and aromatic oils as flavoring agents. The formulation may also contain suitable propellant(s).

For ophthalmic applications, the therapeutic compound is formulated into solutions, suspensions and ointments appropriate for use in the eye. For opthalmic formulations, see Mitra (ed.), Ophthalmic Drug Delivery Systems, Marcel Dekker, Inc., New York, N.Y. (1993) and also Havener, W. H., Ocular Pharmacology, C.V. Mosby Co., St. Louis (1983).

Useful dosages of the compounds of formula I can be determined by comparing their in vitro activity and in vivo activity in animal models. Methods for the extrapolation of effective dosages in mice and other animals, to humans are known to the art; for example, see U.S. Pat. No. 4,938,949. The amount of the substance required for use in treatment will vary not only with the particular salt selected but also with the route of administration, the nature of the condition being treated and the age and condition of the patient and will be ultimately at the discretion of the attendant physician or clinician.

In general, a suitable dose for nuclear medicine (for example, using a radioactive imaging agent) will be in the range of from about 0.1 μg/patient to about 1000 μg/patient, from about 0.5 to about 500 μg/patient or from 1 μg/patient to about 100 μg/patient.

A suitable dose for imaging medicine (for example, using a paramagnetic imaging agent) will be in the range of from about 0.1 mg/patient to about 100 mg/patient, from about 0.5 to about 50 mg/patient or from 1 mg/patient to about 10 mg/patient.

For therapeutic applications, a suitable dose will be in the range of from about 0.05 picograms/kilogram to about 100 mg/kg, from about 10 to about 75 mg/kg of bodyweight per day, such as 3 to about 50 mg per kilogram body weight of the recipient per day, preferably in the range of 6 to 90 mg/kg/day, most preferably in the range of 15 to 60 mg/kg/day. The substance is conveniently administered in unit dosage form; for example, containing 5 to 1000 mg, conveniently 10 to 750 mg, most conveniently, 50 to 500 mg of active ingredient per unit dosage form.

Ideally, the substance should be administered to achieve peak plasma concentrations of from about 0.05 to about 100 μM, preferably, about 1 to 50 μM, most preferably, about 2 to about 30 μM. This may be achieved, for example, by the intravenous injection of a 0.005 to 10% solution of the substance, optionally in saline or orally administered as a bolus containing about 0.5-250 mg of the substance. Desirable blood levels may be maintained by continuous infusion to provide about 0.01-5.0 mg/kg/hr or by intermittent infusions containing about 0.4-15 mg/kg of the substance.

The substance may conveniently be presented in a single dose or as divided doses administered at appropriate intervals, for example, as two, three, four or more sub-doses per day.

The cobalamin conjugates may be administered orally in combination with a pharmaceutically acceptable vehicle such as an inert diluent or an edible carrier. They may be enclosed in hard or soft shell gelatin capsules, may be compressed into tablets or may be incorporated directly with the food of the patient's diet. For oral therapeutic administration, the substance may be combined with one or more excipients and used in the form of ingestible tablets, buccal tablets, troches, capsules, elixirs, suspensions, syrups, wafers and the like. Such compositions and preparations should contain at least 0.1% of the substance. The percentage of the compositions and preparations may, of course, be varied and may conveniently be between about 2 to about 60% of the weight of a given unit dosage form. The amount of substance in such therapeutically useful compositions is such that an effective dosage level will be obtained.

Tablets, troches, pills, capsules and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the substance may be incorporated into sustained-release preparations and devices.

Sublingual tablets are designed to dissolve very rapidly. Examples of such formulations include ergotamine tartrate, isosorbide dinitrate, isoproterenol HCl. The formulation of these tablets contain, in addition to the drug, a limited number of soluble excipients, usually lactose and powdered sucrose, but occasionally dextrose and mannitol. The process of making sublingual tablets involves moistening the blended powder components with an alcohol-water solvent system containing approximately 60% alcohol and 40% water.

In addition to the cobalamin conjugate, the prototype formulation for sublingual tablets may contain a binder such as povidone or HPMC, diluents such as lactose, mannitol, starch or cellulose, a disintegrant such as pregelatinized or modified starch, lubricants such as magnesium stearate, stearic acid or hydrogenated vegetable oil, a sweetener such as saccharin or sucrose and suitable flavoring and coloring agents.

XII. Controlled Release Formulations

In one embodiment, the agent and carrier are administered in a slow release formulation that can be a degradable or nondegradable polymer, hydrogel or ganogel or other physical construct that modifies the bioabsorption, half life or biodegradation of the cobalamin or compound of Formula I/diagnostic or therapeutic agent/cobalamin transport protein, such as an implant, bolus, microparticle, microsphere, nanoparticle or nanosphere. The controlled release formulation can be a material that is painted or otherwise applied onto the afflicted site, either internally or externally. In one embodiment, the invention provides a biodegradable bolus or implant that is inserted into the pocket created by surgical resection of a tumor or directly into the tumor itself. In another example, the controlled release formulation can be applied to a psoriatic lesion, eczema, atopic dermatitis, lichen planus, wart, pemphigus vulgaris, actinic keratosis, basal cell carcinoma or squamous cell carcinoma. The controlled release formulation can likewise be applied to a blood vessel to treat or prevent restenosis, retinopathies or atherosclerosis. The controlled release formulation with appropriated selected imaging agent can be used to coat a transplanted organ or tissue to prevent rejection. It can alternatively be implanted or otherwise applied near the site of rheumatoid arthritis.

The field of biodegradable polymers has developed rapidly since the synthesis and biodegradability of polylactic acid was first reported in 1966 by Kulkarni et al. “Polylactic acid for surgical implants,” Arch. Surg., 93, 839. Several other polymers are now known to biodegrade, such as polyanhydrides and polyorthoesters, which take advantage of labile backbone linkages (see: Domb et al. Macromolecules. 22, 3200, 1989; and Heller et al. Biodegradable Polymers as Drug Delivery Systems, Dekker, NY: 1990). Several polymers which degrade into naturally occurring materials have also been described, such as crosslinking gelatin, hyaluronic acid (della Valle et al. U.S. Pat. No. 4,987,744 and U.S. Pat. No. 4,957,744) and polyaminoacids (Miyake et al., 1974), which spurred the usage of polyesters by Holland et al. Controlled Release. 4, 155, 1986 and alph-hydroxy acids (i.e. lactic acid and glycolic acid), which remain the most widely used biodegradable materials for applications ranging from closure devices (sutures and staples) to drug delivery systems (Smith et al. U.S. Pat. No. 4,741,337; Spilizeqski et al. J. Control. Rel., 2, 197, 1985).

These polymers can be tailored to degrade at a desired rate and with a desired kinetics by selecting the appropriate monomers, method of preparation and molecular weight. Differences in crystallinity of the monomer can alter the polymeric degradation rate. Due to the relatively hydrophobic nature of most polymers, actual mass loss can begin with the oligomeric fragments that are small enough to be water soluble; hence, even the initial molecular weight can influence the degradation rate.

Hydrogels can be used in controlled release formulations. Such polymers are formed from macromers with a polymerizable, non-degradable, region that is separated by at least one degradable region. For example, the water soluble, non-degradable, region can form the central core of the macromer and have at least two degradable regions which are attached to the core, such that upon degradation, the non-degradable regions (in particular a polymerized gel) are separated. Specifically, as disclosed in U.S. Pat. No. 5,626,863 to Hubbell et al., the macromers are PEG-oligoglycolyl-acrylates, with the appropriate end caps to permit rapid polymerization and gelation. Acrylates can be polymerized readily by several initiating systems such as eosin dye, ultraviolet or visible light. The polyethyleneglycol (PEG) is highly hydrophilic and biocompatible. The oligoglycolic acid is a poly(a-hydroxy acid) which can be readily degraded by hydrolysis of the ester linkage into glycolic acid, a nontoxic metabolite. Other chain extensions include polylactic acid, polycaprolactone, polyorthoesters, polyanhydrides and polypeptides. This entire network can be gelled into a biodegradable network that can be used to entrap and homogeneously disperse water-soluble drugs for delivery at a controlled rate. Further, the gel can entrap particulate suspensions of water-insoluble drugs. (See also: U.S. Pat. No. 4,591,496 to Cohen et al. (Process for Making Systems for the Controlled Release of Macromolecules); U.S. Pat. No. 5,545,442 to Van Savage et al. (Method for Using a Radiation Cured Drug Release Controlling Membrane); U.S. Pat. No. 5,330,768 to Park et al. (Controlled Drug Delivery Using Polymer/Pluronic Blends); U.S. Pat. No. 5,122,367 to Ron et al. (Polyanhydride Bioerodible Controlled Release Implants for Administration of Stabilized Growth Hormone); U.S. Pat. No. 5,545,409 to Laurencin et al. (Delivery System for Controlled Release of Bioactive Factors); U.S. Pat. No. 5,629,009 to Laurencin et al (Delivery System for Controlled Release of Bioactive Factors).

Alternatively, delivery of biologically active substances, both in vitro and in vivo, via encapsulation has been well described in the prior art. U.S. Pat. No. 4,352,883 to Lim et al. entitled “Encapsulation of Biological Material” discloses the encapsulation of proteins within a membrane by suspending the protein in an aqueous medium containing a water-soluble gum that can be reversibly gelled to form the suspension into droplets. These droplets can be gelled further into discrete, shape-retaining, water insoluble temporary capsules with the aid of a solution of multivalent cations. The temporary capsules then can be further wrapped by an ionically cross-linking surface layer to form a semipermeable membrane around the capsules that is permeable to small molecules but impermeable to larger molecules. Microencapsulations of glycoproteins have also been well described. U.S. Pat. No. 4,324,683 to Lim et al. entitled “Encapsulation of Labile Biological Material” encapsulates a glycoprotein by a two-step interfacial polymerization process to form capsules with well-controlled porosity. The microcapsules serve to protect the active substances from attack by microorganisms and from any immunological response. U.S. Pat. No. 5,718,921 to Mathiowitz et al. (Microspheres Comprising Polymer and Drug Dispersed There Within) discloses a method to encapsulate relatively temperature-labile drugs into a microsphere.

Several methods have been developed to reversibly encapsulate biologically active substances: One that can be applied both to in vitro and in vivo studies has been described in U.S. Pat. No. 4,900,556 by Wheatley et al. entitled “System for Delayed and Pulsed Release of Biologically-Active Substances.” In this disclosed system, the biologically-active substance can be released either at a constant rate over a period of time or in discrete pulses. The biologically active materials are entrapped within liposomes encapsulated within semipermeable microcapsules or permeable polymeric matrix. Release of the desired materials is governed by the permeability of both the liposome and the surrounding matrix (the matrix integrity is directly proportional to the liposome integrity); the permeability of the liposome can be engineered by modifying the composition and the method for making the liposome to produce liposome that are sensitive to specific stimuli such as temperature, pH or light. For example, by including a phospholipase that degrades the liposome within some or all of the liposomes or the surrounding matrix, the liposome can be destabilized and broken down over a period of time. Other systems have been developed, e.g. U.S. Pat. No. 4,933,185 by Wheatley et al., which utilize a core made up of a polymer (such as an ionically cross-linked polysaccharide with calcium alginate or chitin) around which there is an ionically bound skin (such as a polycationic skin of poly-L-lysine) whose integrity is dependent on the core polymer. With an impermeable skin, when the core polymer can be degraded by enzymes (such as alginase from the bacteria, chitinase or hydrolase), there is a sudden release of biologically active substance from the core. Alternatively, the skin can be partially permeable for a gradual release of drug upon degradation of the core.

Nanoparticles are especially useful in the delivery of drugs parenterally or intravenously such that the delivery device is small with a long circulating half-life. A number of injectable drug delivery systems have been investigated, including microcapsules, microparticles, liposomes and emulsions. The major obstacle for these delivery systems is the rapid clearance of the materials from the blood stream by the macrophages of the reticuloendothelial system (RES). For example, polystyrene particles as small as sixty nanometers in diameter are cleared from the blood within two to three minutes. Liposomal drug delivery systems have also been extensively studied for this application because they were expected to freely circulate in the blood. Coating of the liposomes with poly(ethylene glycol) (PEG) increased the half-life of the carriers due to PEG's hydrophobic chains which reduced its protein absorption and thus its RES uptake. U.S. Pat. No. 5,543,158 to Gref et al. (Biodegradable Injectable Nanoparticles) describes a carrier system specifically targeted towards carriers suitable for intravenous delivery with a controlled release mechanism with modified polyglycols.

U.S. Pat. No. 5,626,862, U.S. Pat. No. 5,651,986 and U.S. Pat. No. 5,846,565 to Brem et al. (Controlled Local Delivery of Chemotherapeutic Agents for Treating Solid Tumors) discloses the use of these carriers for the specific delivery of chemotherapeutic agents to increase bioavailability. Therefore, the devices act as reservoirs that release drugs over an extended period of time while at the same time preserves the bioactivity and bioavailability of the agent. U.S. Pat. No. 5,286,763 to Gerhard et al. (Bioerodible Polymers for Drug Delivery in Bone) further discloses that bioerodible polymers can be used to deliver chemotherapeutic agents directly into the bone. Cohen et al. U.S. Pat. No. 5,562,099 (Polymeric Microparticles Containing Agents for Imaging) discusses the usage of these carriers as contrast agents. The polymeric microparticle is filled with contrast agents for enhanced imaging.

Books describing methods of controlled delivery that are appropriate for the delivery of the cobalamin or compound of Formula I/imaging agents of the present invention include: Robert S. Langer, Donald L. Wise, editors; Medical applications of controlled release (Volumes 1 and 2); Boca Raton, Fla.: CRC Press, 1984; and William J. M. Hrushesky, Robert Langer and Felix Theeuwes, editors; Temporal control of drug delivery (series); New York: New York Academy of Sciences, 1991.

The invention will now be illustrated by the following non-limiting Examples.

### EXAMPLES

### Example 1

### Preparation of Cyanocobalamin-b-(4-aminobutyl)amide

A mixture containing cyanocobalamin-b-carboxylic acid (1.0 g, 0.6 mmol), hydroxybenzotriazole (0.81 g, 6 mmol) and 1,4-diaminobutane dihydrochloride (4.8 g, 30 mmol) in 100 ml of water was adjusted to pH 7.8. 1-Ethyl-3-(3′-dimethylaminopropyl)-carbodiimide (1.26 g, 6.6 mmol) was then added, the pH was adjusted to 6.4 and the reaction stirred at room temperature for 24 h. TLC on silica gel using n-butanol-acetic acid water (5:2:3) showed the reaction to be complete. Cyanocobalamin-b-(4-aminobutyl)amide was extracted into 92% aqueous phenol and the phenol layer was washed several times with equal volumes of water. To the phenol extract were added 3 volumes of diethylether and 1 volume of acetone. The desired cobalamin was removed from the organic phase by several extractions with water. The combined aqueous layers were extracted three times with diethylether to remove residual phenol, concentrated to approximately 20 ml in vacuo and crystallized from aqueous acetone. Yield 955 mg, 92%.

### Example 2

### Preparation of Methylcobalamin-b-(4-aminobutyl)amide

Methylcobalamin-b-carboxylic acid (1.0 g, 0.6 mmol) was reacted with diaminobutane dihydrochloride as described above for the cyano derivative. The cobalamin was purified by extraction through phenol (see above) and the resulting aqueous solution was concentrated in vacuo. This solution was chromatographed on AG1-X2 200-400 mesh in the acetate form (20.times.2.5 cm) and the pass through collected. The pass through was concentrated to approximately 20 ml and the desired cobalamin crystallized from aqueous acetone. Yield 920 mg, 88%. Unreacted methylcobalamin-b-carboxylic acid was eluted with 1M acetic acid, concentrated and crystallized from aqueous acetone. Yield 60 mg, 6%.

### Example 3

### Preparation of Adenosylcobalamin-b-(4-aminobutyl)amide

Adenosylcobalamin-b-carboxylic acid (500 mg, 0.3 mmol) was reacted with diaminobutane dihydrochloride (2.4 mg, 15 mmol) as described above. The cobalamin was purified by extraction through phenol (see above). The resulting aqueous solution was concentrated in vacuo and applied to AG-50 X2, 200-400 mesh, in the hydrogen form (20.times.25 cm). The column was washed thoroughly with water to remove hydroxybenzotriazole and the desired cobalamin eluted with 1M ammonium hydroxide. After an additional extraction through phenol, adenosylcobalamin-b-(4-aminobutyl)amide was isolated as a glass. Yield 366 mg, 77%.

### Example 4

### Proposed Preparation of Cyanocobalamin-b-(4-aminobutyl)amide-, Ciprofloxacin-, Levofloxacin-, Ofloxacin- and Sparfloxacin-Cobalamin Conjugates

A mixture containing cyanocobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and I volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 5

### Proposed Preparation of Methylcobalamin-b-(4-aminobutyl)amide- Ciprofloxacin-, Levofloxacin-, Ofloxacin- and Sparfloxacin-Cobalamin Conjugates

A mixture containing methylcobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-343′ dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 6

### Proposed Preparation of Adenosylcobalamin-b-(4-aminobutyl)amide- Ciprofloxacin-, Levofloxacin-, Ofloxacin- and Sparfloxacin-Cobalamin Conjugates

A mixture containing adenosylcobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 7

### Proposed preparation of Cyanocobalamin-b-(4-aminobutyl)amide- Lisinopril-, Fosinopril Sodium-, Enalaprilat-, and Captopril-Cobalamin Conjugates

A mixture containing cyanocobalamin-b-(4-aminobutyl)amide (0.6 mmol), hydroxybenzotriazole (6 mmol) and the cardiovascular agent (e.g., Lisinopril, Fosinopril Sodium, Enalaprilat, or Captopril) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone.

The desired product is removed from the organic phase by several extractions with water. The combined aqueous layers are extracted three times with diethylether to remove residual phenol, concentrated to approximately 20 ml in vacuo and crystallized from aqueous acetone.

### Example 8

### Proposed preparation of Methylcobalamin-b-(4-aminobutyl)amide- Lisinopril-, Fosinopril Sodium-, Enalaprilat-, and Captopril-Cobalamin Conjugates

A mixture containing methylcobalamin-b-(4-aminobutyl)amide (0.6 mmol), hydroxybenzotriazole (6 mmol) and the cardiovascular agent (e.g., Lisinopril, Fosinopril Sodium, Enalaprilat, or Captopril) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 9

### Proposed preparation of Adenosylcobalamin-b-(4-aminobutyl)amide- Lisinopril-, Fosinopril Sodium-, Enalaprilat-, and Captopril-Cobalamin Conjugates

A mixture containing adenosylcobalamin-b-(4-aminobutyl)amide (0.6 mmol), hydroxybenzotriazole (6 mmol) and the cardiovascular agent (e.g., Lisinopril, Fosinopril Sodium, Enalaprilat, or Captopril) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 10

### Preparation of Cyanocobalamin-b-poly-L-lysine)amide

Two preparations of poly-L-lysine hydrobromide, one containing approximately 8 residues and a second one containing about 11 residues were separately reacted with cyanocobalamin-1-carboxylic acid. To each polymer (500 mg) dissolved in 20 mL of water was added 150 mg (0.1 mmol) of cyanocobalamin-1-carboxylic acid, 338 mg (2.5 mmol) of hydroxybenzotriazole and 480 mg (2.5 mmol) of 1-ethyl-3(3-dimethyl-aminopropyl) carbodiimide. The pH was adjusted to 9 with 1N NaOH and the reaction mixtures were stirred at room temperature for 2-3 h. They were purified on G-10 sephadex: the sizing columns (3×40 cm) were eluted with water and 1.5 mL fractions collected. The fractions showing the presence of the cobalamin (OD at 550 mm) and the presence of polylysine (ninhydrin positive) were pooled and freeze-dried.

### Example 11

### Proposed Preparation of Cyanocobalamin-b-(polylysine)amide-, Ciprofloxacin-, Levofloxacin-, Ofloxacin- and Sparfloxacin-Conjugates

A mixture containing cyanocobalamin-b-(polylysine)amide (0.6 mmol), hydroxybenzotriazole (0.81 g, 6 mmol) and the antibiotic (e.g. Ciprofloxacin, Levofloxacin, Ofloxacin or Sparfloxacin) (30 mmol) in 100 ml of water is adjusted to pH 7.8. 1-Ethyl-3-(3′-dimethylaminopropyl)carbodiimide (1.26 g, 6.6 mmol) is then added, the pH is adjusted to 6.4 and the reaction is stirred at room temperature for 24 h. TLC on silica gel using n-butanol-acetic acid water (5:2:3) shows the reaction to be complete. The product is purified on G-10 sephadex; the sizing columns (3×40 cm) are eluted with water and 1-5 mL fractions are collected. The fractions showing the presence of cobalamin (OD at 550 mm) and the presence of polylysine (ninhydrin positive) are pooled and freeze-dried.

### Example 12

### Proposed Preparation of Cyanocobalamin-b-(polylysine)amide- Lisinopril-, Fosinopril Sodium-, Enalaprilat-, and Captopril-Conjugates

A mixture containing cyanocobalamin-b-(polylysine)amide, hydroxybenzotriazole (0.81 g, 6 mmol) and the cardiovascular agent (e.g., Lisinopril, Fosinopril Sodium, Enalaprilat, or Captopril) (30 mmol) in 100 ml of water is adjusted to pH 7.8. 1-Ethyl-3-(3′-dimethylaminopropyl)carbodiimide (1.26 g, 6.6 mmol) is then added, the pH is adjusted to 6.4 and the reaction is stirred at room temperature for 24 h. TLC on silica gel using n-butanol-acetic acid water (5:2:3) shows the reaction to be complete. The reaction mixture is purified on G-10 sephadex: the sizing columns (3×40 cm) are eluted with water and 1.5 mL fractions collected. The fractions showing the presence of the cobalamin (OD at 550 mm) and the presence of polylysine (ninhydrin positive) are pooled and freeze-dried.

### Example 13

### Cyanocobalamin-b-(4-aminobutyl)amide DTPA

Cyanocobalamin-b-(4-aminobutyl) amide (500 mg), 0.3 mmol) was dissolved in 30 ml saturated sodium bicarbonate and treated with solid DTPA dianhydride (1.2 g, 3.4 mmol). The progress of the reaction was monitored by TLC on PEI plates using n-butanol-acetic acid-water (5:2:3) as the solvent. After 30 min incubation at room temperature a second 1.2 g of the dianhydride was added. After two additional additions of dianhydride with adjustments of the pH to 8.2 the reaction mixture was incubated overnight. Cyanocobalamin-DPTA adduct was then extracted into 92% aqueous phenol and purified as described above. The preparation was evaporated to dryness in vacuo and isolated as a glass. Yield 460 mg, 77%. The cyanocobalamin-DTPA adduct behaves as a polyanion on paper electrophoresis in 0.1 M sodium phosphate buffer pH 7.1.

### Example 14

### Methylcobalamin-b-(4-aminobutyl)amide DTPA

Methylcobalamin-b-4-aminobutyl)amide (500 mg, 0.3 mmol) was dissolved in 30 ml saturated sodium bicarbonate and reacted with solid DTPA dianhydride as described above. The methyl cobalamin-DTPA adduct was purified by extraction through phenol, evaporated to dryness in vacuo and isolated as a glass. Yield 600 mg, 96%.

### Example 15

### Adenosylcobalamin-b-(4-aminobutyl)amide DTPA

Adenosylcobalamin-b-(4-aminobutyl)amide (366 mg, 0.23 mmol) was dissolved in 30 ml saturated sodium bicarbonate and treated with solid DTPA dianhydride (1.0 g, 2.8 mmol) as described above. The cobalamin was purified through phenol (see above). The resulting aqueous solution was concentrated and applied to AG-50 X2, 200-400 mesh, in the hydrogen form (6.0×2.5 cm), the column was washed with water and the desired cobalamin eluted with 0.1 M ammonium hydroxide. The solution was evaporated to dryness in vacuo and adenosylcobalamin-b-(4-aminobutyl)amide DTPA isolated as a glass. Yield 400 mg, 80%.

### Example 16

### Chelation of Radionuclides

Under dim light, 1000 μg of methyl-, adenosyl-, and cyanocobalamin-b-(4-aminobutyl)amide-DTPA were separately dissolved in 200 μL of normal saline. Next, 500 μCi of Indium-111 or 250 μCi of Gadolinium-153 were added to the cobalamin-DTPA solutions. The reactions were carried out at room temperature and room air. For the chelation of technetium, the dissolved cobalamin DTPA complexes were separately placed into sealed 2 ml vials. Next, 200 μL of stannous chloride solution (1000 μg/ml normal saline) was added to each vial. The vials were purged with nitrogen gas for 5 minutes. After this time, 1-5 μCi of Technetium-99m was added to the N₂ purged vials. Each vial underwent further nitrogen purging for 5 minutes. All chelation reactions were mixed gently for 5 minutes.

Control mixtures of 1000 μg of cyanocobalamin were dissolved in 200 μL of normal saline. Cyanocobalamin was mixed with Tc-99m at room temperature and room air, as well as within nitrogen purged vials containing 200 μL of the described stannous chloride solution. Additionally, the cobalamin-DTPA complexes underwent Tc-99m labeling in open vials at room air in the absence of the stannous chloride.

### Example 17

### Synthesis of Daunorubicin- and Doxorubicin-Cobalamin Conjugates

Modification of the carbohydrate moiety (daunosamine) of daunorubicin (1) with L-leucine can be accomplished by reacting daunorubicin HCl (0.5 g) in 100 mL borate buffer pH=10 (containing KCl) with L-leucine-carboxyanhydride (1 mmole in 5 mL acetone) at 0° C. under nitrogen. After reaction for 5 minutes at 0° C., the mixture can be acidified to pH 3.5 with H₂SO₄, stirred for 15 minutes and adjusted to pH=7 to give the desired L-leucyl daunorubicin (2). Reaction of (2) with a cobalamin-mono or dicarboxylic acid in the presence of a water-soluble carbodiimide and hydroxybenzotriazole will yield the daunorubicin-cobalamin conjugate (3). These conjugates can be isolated via the usual phenol extraction, extensive washing of the phenol phase with water and finally displacing the cobalamin-conjugates from the phenol phase into water by the addition of acetone and diethyl ether.

Modification of doxorubicin should be similar (Ger. Patent 1,813,518, Jul. 10, 1969; Chem Abstracts, 71, 91866 (1969)). D. Deprez-Decampaneere, M. Mosquelier, R. Bourain and A. Trosect, Curr. Chemother. Proc., Int. Congr. Chemother., 10th, p. 1242 (1978) have found that N-(L-leucyl) daunorubicin but not the D isomer was hydrolyzed in vivo to regenerate daunorubicin. See, “Doxorubicin, Anticancer Antibiotics,” Federico Arcamone, Medicinal Chemistry, Vol. 17, Academic Press, 1981.

### Example 18

### Synthesis of Peptide Nucleic Acid (PNA)-Nuclear Localization Peptide (TAT) Chimera

The nuclear localization signal peptide TAT (ryr-Gly-Arg-Lys-Lys-Arg-Arg-Gln-Arg Arg-Arg) is synthesized as a peptide amine by a solid-phase method on Rink (4-2′,4′-dimethozyphenyl-Fmoc-aminomethyl-phenoxy) co-polystyrene resin (0.1 mmole) with N^{α}-Fmoc L-amino acids (Calbiochem-Novabiochem Corp., San Diego, Calif.). Ten equivalents (1.0 mmole) of each Fmoc-L-amino acid was activated with PyBop/HoBt/4-Methylmorpholine and coupled to the resin-linked peptide chain in 1-methyl-2-pyrrolidinone (NMP) for 2 h following deprotection of each N^{α}-Fmoc protecting group with 20% piperidine in NMP for 30 minutes.

An anti-viral peptide nucleic acid (PNA) is sequentially added to the free amino group of the resin-bound TAT peptide, starting with the first base at the 3′-end of the PNA molecule. The synthesis of the PNA uses Fmoc-N-(2-aminoethyl) glycyl PNA monomers on an Expidite 8909 Nucleic Acid Synthesizer according to cycle protocols developed by the manufacturer (Perseptive Biosystems, Inc., Foster City, Calif.). The exocyclic amines of the bases adenine, guanine, and cytosine of each Fmoc-PNA monomer are protected with the blocking group benzhydryloxycarbonyl).

The Fmoc group of each PNA monomer is removed by treatment with 20% piperidine in dimethylformamide (DMF) for 15 min, followed by activation and coupling of the next PNA monomer (5 equivalents) with HATU (4.5 equiv.), 2,6-lutidine (7.5 equiv.) and diisopropylethylamine (5 equiv.) for 30 minutes. Addition of an AEEA [2(2-aminoethoxy) ethoxy]acetic acid monomer is added to the 5′-end of the synthesized PNA as a spacer group before linkage of the vitamin B₁₂ molecule.

### Example 19

### Synthesis of Vitamin B₁₂ (B carboxylate form) to PNA-TAT chimera

Vitamin B₁₂ (free carboxylate form) is added to the amino terminal groups of the AEEA-PNA-TAT chimera by activation of vitamin B₁₂'s carboxylic acid with PyBop/HoBt/4-Methymorpholine in DMF, and subsequent coupling of the mixture in DMF for 2 hours.

After coupling of the Vitamin B₁₂, the vitamin B₁₂-PNA-TAT chimera is deprotected and removed form the rink-resin support by treatment with a mixture of 90% TFA/5.0% water/2.5% ethanedithiol/2.5% thioanisole for 90 min at room temperature. The deprotected crude product is washed and separated by precipitation in 3×50 volumes of cold methyl t-butyl ether, and purified by reverse phase HPLC on Vydac C18 column (2.1)×25 cm) in 0.1% TFA/water with a 60 min gradient of 10%-89% acetonitrile in 0.1% TFA. The composition of the vitamin B₁₂-PNA-TAT product is analyzed by Electrospray Ionization (ESI) Mass Analysis on a PE SCIEX API 165 Biospectrometer (Applied Biosystems, Inc.)

### Example 20

### Interaction with Cobalamin Transport Protein

Under dim light, 1000 μg of the non-labeled methyl-, adenosyl-, and cyanocobalamin-b-(4-aminobutyl)amide-DTPA, as well as 1000 μg of cyanocobalamin and DTPA (Sigma, St. Louis, Mo. 63178), were separately dissolved in 10 mL of normal saline at room temperature. Each of the five 1000 μg/10 ml samples were stored in sealed, aluminum-wrapped 10 ml vials to prevent exposure to light. No buffers were added to the solutions. The pH of the solutions, measured by a Beckman 40 pH meter (Beckman Instruments, Fullerton, Calif.): Cyanocobalamin=5.75, DTPA=3.78; cyano, methyl and adenosylcobalamin-DTPA analogues were 5.75, 6.10, and 6.19, respectively.

To assess in vitro binding to Intrinsic Factor (IF) and Transcobalamins (TC), the intrinsic factor blocking antibody (IFBA) and Unsaturated vitamin B₁₂ Binding Capacity (UBBC) assays were performed with serum randomly obtained from five patients being evaluated for pernicious anemia at the Mayo Clinic. The IFBA and UBBC assays were first performed for clinical purposes as previously described by V. F. Fairbanks et al., Mayo Clin. Proc. 58, 203 (1983); Intrinsic Factor Blocking Antibody (⁵⁷Co) Radioassay-Package insert, Diagnostic Products Corp.; D. Grossowicz et al., Proc. Exp. Biol. 109, 604 (1962) and C. Gottlieb et al., Blood 6 (1965).

Next, the serum from the same five patients underwent modified IFBA and UBBC assays. Specifically, 1 μL of the five previously described solutions were separately incubated with purified IF or serum, to potentially saturate all IF and TC-binding sites. After incubation for 20 minutes at room temperature and for another 20 minutes at 4° C., 500 μL of the stock (1000 μg/l) Cobalt-57-cyanocobalamin (Mallinckrodt Medical, Inc., St Louis, Mo. 63134) solution was added and the usual IFBA and UBBC protocols were then followed. All supernatant activity was counted for four minutes on a gamma counter (Micromedix 10/20, Huntsville, Ala. 35805).

The IFBA assay demonstrated that DTPA does not significantly bind to IF (values less than the negative reference), whereas cyanocobalamin and the cobalamin-DTPA analogs do, in varying degrees, competitively inhibit Co-57 cyanocobalamin from binding to intrinsic factor. By using the counts of the Clinical run divided into the counts of the five solutions, the efficacy of binding to intrinsic factor can be estimated. The averaged percent binding of the five solutions to IF was: cyanocobalamin=92.5%; methylcobalamin-b-(4-aminobutyl)-amide-DTPA=63.2%; cyanocobalamin-b-(4-aminobutyl)-amide-DTPA=52.9%; adenosylcobalamin-b-(4-aminobutyl)-amide-DTPA=41.0% and 0.8% for DTPA. This is in contrast to the disclosure in Houts (U.S. Pat. No. 4,465,775) that the (b)-monocarboxylic acid of vitamin B₁₂ and its radioiodinated derivative exhibit very low binding to IF.

Likewise the averaged percent binding of the five solutions to the transcobalamin proteins was: cyanocobalamin=100%, methylcobalamin-b-(4-aminobutyl)amide-DTPA=94.0%, adenosylcobalamin-b-(4-aminobutyl)amide-DTPA=90.4%, cyanocobalamin-b-4-aminobutyl)amide-DTPA=66.4% and 3.6% for DTPA.

Thus, the attachment of DTPA to vitamin B₁₂ does alter its binding to the carrier proteins. As expected, non-labeled cyanocobalamin had the greatest affinity for IF and the transcobalamin proteins. Methylcobalamin-b-(4-aminobutyl)amide-DTPA was next; followed by adenosylcobalamin-b-(4-aminobutyl)amide-DTPA, and finally cyanocobalamin-b-4-aminobutyl)amide-DTPA. There was also some nonspecific binding of DTPA to the carrier proteins (0.8% and 3.6%).

### Example 21

### Coadministration Dosage Regimes

The term “active ingredient” as used below is vitamin B₁₂ or a compound of Formula I, linked to a diagnostic, therapeutic or other material, administered in any ratio that achieves the desired result. In one embodiment the ratio is one molecule of the vitamin B₁₂ or a compound of Formula I to at least one molecule of cobalamin transport protein. In an alternate embodiment of the invention, the ratio is one molecule of the vitamin B₁₂ or a compound of Formula I to at least one molecule of cobalamin transport protein, and preferably with an excess of cobalamin transport protein, for example, 1.5, 2, 3, 4, 5, or more times excess of cobalamin transport protein. In another embodiment of the invention, the ratio is at least one molecule of the vitamin B₁₂ or a compound of Formula I to one molecule of cobalamin transport protein, and preferably with an excess of vitamin B₁₂ or a compound of Formula I, for example, 1.5, 2, 3, 4, 5, or more times excess of vitamin B₁₂ or a compound of Formula I.

The mixtures are prepared by physically mixing the transport protein with the vitamin B₁₂ or a compound of Formula I linked to a diagnostic, therapeutic or other material prior to formulation in a pharmaceutically acceptable carrier. Alternatively, the mixtures are prepared by simply mixing them separately with the carrier. The active ingredient contains a cobalamin or a compound of Formula I complex that is either administered bound (i.e. either covalently, ionically, datively or via van der Waals attraction), or unbound (i.e. admixed with) to intrinsic factor.

Non-limiting examples, the active ingredient is prepared as pharmaceutical formulations via the following:

Capsules (Hard)

Hard capsules can be prepared by filling standard two-piece hard gelatin capsules with the following mixture using conventional encapsulating equipment:
Active ingredient: 1 mg
Lactose: 125 mg
Talc: 12 mg
Magnesium stearate: 3 mg
   Capsules (Soft)

A mixture of active ingredient in soybean oil can be prepared and injected by means of a positive displacement pump in gelatin to form soft gelatin capsules containing 5 mg of the active ingredient. The capsules can be washed in petroleum ether and dried.

Tablets

Tablets can be prepared by conventional procedures so that each unit will contain:
Active ingredient: 1 mg
Spray dried lactose: 150 mg
Microcrystalline cellulose: 35 mg
Magnesium stearate: 3 mg
   Parenteral

Parenteral composition suitable for intramuscular administration can be prepared so that each mL contains, percentages being by weight:
Active ingredient: 1 mg
Sodium carboxymethyl cellulose: 0.75%
Polysorbate 80: 0.04%
Benzyl alcohol: 0.9%
Sodium chloride: 0.9%
Water for injection Q.S.: 1 mL
   Suspension

An aqueous suspension can be prepared for oral administration so that each 5 mL contain, percentages being by weight:
Active ingredient: 5 mg
Methylcellulose: 5%
Carboxymethyl cellulose: 5%
Syrup: 30%
Polysorbate 80: 0.2%
Sodium saccharin: 2 mg
Cherry flavor: 0.1%
Sodium benzoate: 5 mg
Water Q.S.: 5 mL

The invention has been described with reference to various specific and preferred embodiments and techniques. However, it should be understood that many variations and modifications may be made while remaining within the spirit and scope of the invention.

1. A method for increasing the uptake of cobalamin-bound detectable or therapeutic agent to a host in need thereof comprising providing the cobalamin-bound detectable or therapeutic agent in combination with a cobalamin transport protein.

2. The method of claim 1 wherein the cobalamin transport protein is intrinsic factor, transcobalamin I, transcobalamin II, transcobalamin III, or any combination thereof.

3. The method of claim 1 wherein the cobalamin linked diagnostic or therapeutic conjugated to a cobalamin transport protein is administered via intravenous, parenteral, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, rectal, vaginal or urethral administration including via suppository, percutaneous, nasal spray, surgical implant, internal surgical paint, infusion pump or catheter.

4. The method claim 1 wherein the the cobalamin linked diagnostic or therapeutic conjugated to a cobalamin transport protein is administered to patients that do not have a cobalamin or cobalamin transport protein deficiency.

5. The method of claim 1, wherein the cobalamin conjugate is a compound of the formula:
The compound of Formula I is of the structure: 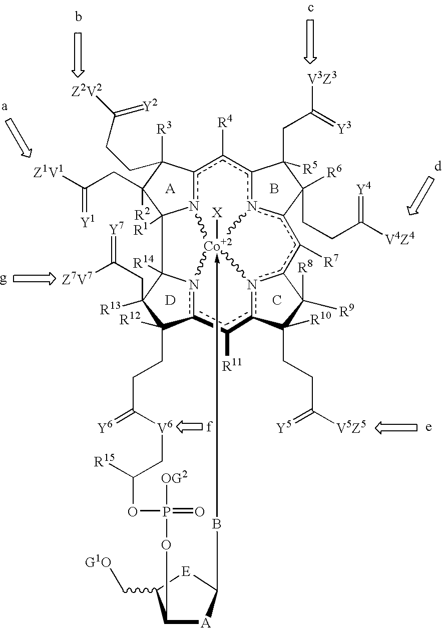
or its enantiomer, diastereomer, salt or prodrug thereof, wherein:
(xviii) the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;
(xix) the dotted line in the chemical structure indicates either a double or single bond such that the double bond does not over-extend the valence of the element (i.e. to give pentavalent carbons) and, in the case of a single bond, the valence is completed with hydrogen
(xx) X is hydrogen, cyano, amino, amido, hydroxyl, adenosyl. L-T, alkyl, alkenyl, alkynyl, cylcoalkyl, aryl, aralkyl, heterocycle, heteroaryl or alkylheteroaryl;
(xxi) B is a divalent heterocycle wherein the radical positions can be within the ring or a substituent to the ring such that at least one radical is on a heteroatom to form a dative bond with cobalt, optionally substituted by L-T;
(xxii) A is O, S, NJ¹, CR¹⁰⁰R¹⁰¹ or C(R¹⁰⁰)V⁸Z⁸;
(xxiii) E is O or S;
(xxiv) G¹ and G² are independently hydrogen, alkyl, acyl, silyl, phosphate, or L-T;
(xxv) Y¹, Y², Y³, Y⁴, Y⁵, Y⁶ and Y⁷ independently are O, S or NJ²;
(xxvi) V¹, V², V³, V⁴, V⁵, V⁶, V⁷ and V⁸ independently are O, S or NJ³; CR¹⁰²R¹⁰³, or a direct bond;
(xxvii) Z¹, Z², Z³, Z⁴, Z⁵, Z⁷ and Z⁸ independently are R¹⁰⁴ or L-T;
(xxviii) each L is independently a direct bond or the residue of a multivalent moiety that does not significantly impair the ability of the compound to bind to a cobalamin transport protein;
(xxix) each T is independently a diagnostic or therapeutic agent;
(xxx) at least one of Z¹, Z², Z³, Z⁴, Z⁵, Z⁷, Z⁸, A, B, G¹, and G² comprises an a nucleic acid sequence useful in antisense technology, a peptide nucleic acid or morpholino nucleic acid;
(xxxi) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xxxii) R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(xxxiii) R¹³ and R¹⁴ optionally can come together to form a pi bond; and
(xxxiv) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

6. The method of claim 1, or 5, wherein the therapeutic is an antibiotic.

7. The method of claim 1 or 5, wherein the therapeutic is useful for the treatment of a disorder associated with abnormal cellular proliferation.

8. The method of claim 1 or 5, wherein therapeutic agent is useful for the treatment of an infectious disease.

9. The method of claim 1 or 5, wherein the therapeutic agent is useful in the treatment of a cardiovascular disorder.

10. The method of claim 1 or 5, wherein the therapeutic agent is a nucleic acid, peptide nucleic acid, morpholino nucleic acid, or other material that affects gene expression.

11. The method of claim 1 or 5, wherein the detectable agent is useful in radioimaging.

12. The method of claim 1 or 5, wherein the detectable agent is a radionuclide or paramagnetic metal atom.

13. The method of claim 1, wherein the cobalamin transport protein is linked directly or by a linker to a detectable radionuclide, or paramagnetic metal atom.

14. The method of claim 4 wherein a detectable agent comprising a metallic radionuclide or paramagnetic metal atom is linked to the cobalamin.

15. The method of claim 14 wherein the detectable chelating group is DPTA.

16. The method of claim 14 wherein the metallic radionuclide or paramagnetic metal atom is Technetium-99m, Indium-111, or Gadolinium-157.

17. The method of claim 13 wherein the detectable radionuclide is a non-metallic radionuclide.

18. The method of claim 17 wherein the non-metallic radionuclide is Carbon-1, Fluorine-18, Bromine-76, Iodine-123, or Iodine-124.

19. A composition comprising a cobalamin-bound detectable or therapeutic agent in combination with a cobalamin transport protein.

20. The composition of claim 19 wherein the cobalamin transport protein is intrinsic factor, transcobalamin I, transcobalamin II, transcobalamin III, or any combination thereof.

21. The composition of claim 19 wherein the cobalamin linked diagnostic or therapeutic conjugated to a cobalamin transport protein is administered via intravenous, parenteral, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, rectal, vaginal or urethral administration including via suppository, percutaneous, nasal spray, surgical implant, internal surgical paint, infusion pump or catheter.

22. The method claim 19 wherein the the cobalamin linked diagnostic or therapeutic conjugated to a cobalamin transport protein is administered to patients that do not have a cobalamin or cobalamin transport protein deficiency.

23. The method of claim 19, wherein the cobalamin conjugate is a compound of the formula:
The compound of Formula I is of the structure: 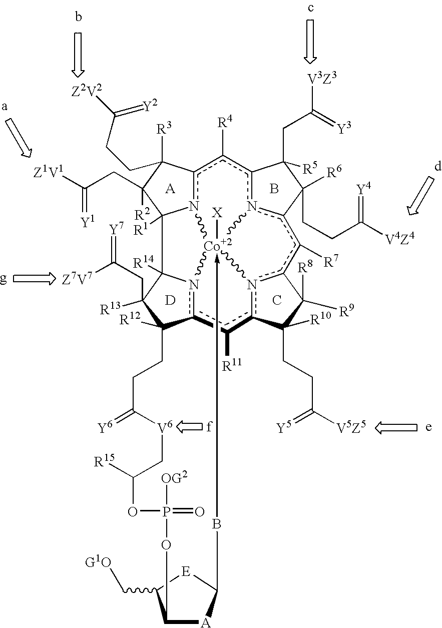
or its enantiomer, diastereomer, salt or prodrug thereof, wherein:
(xxxv) the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;
(xxxvi) the dotted line in the chemical structure indicates either a double or single bond such that the double bond does not over-extend the valence of the element (i.e. to give pentavalent carbons) and, in the case of a single bond, the valence is completed with hydrogen
(xxxvii) X is hydrogen, cyano, amino, amido, hydroxyl, adenosyl L-T, alkyl, alkenyl, alkynyl, cylcoalkyl, aryl, aralkyl, heterocycle, heteroaryl or alkylheteroaryl;
(xxxviii) B is a divalent heterocycle wherein the radical positions can be within the ring or a substituent to the ring such that at least one radical is on a heteroatom to form a dative bond with cobalt, optionally substituted by L-T;
(xxxix) A is O, S, NJ¹, CR¹⁰⁰R¹⁰¹ or C(R¹⁰⁰)V⁸Z⁸;
(xl) E is O or S;
(xli) G¹ and G² are independently hydrogen, alkyl, acyl, silyl, phosphate, or L-T;
(xlii) Y¹, Y², Y³, Y⁴ Y⁵, Y⁶ and Y⁷ independently are O, S or NJ²;
(xliii) V¹, V², V³, V⁴, V⁵, V⁶, V⁷ and V⁸ independently are O, S or NJ³; CR¹⁰²R¹⁰³, or a direct bond;
(xliv) Z¹, Z², Z³, Z⁴, Z⁵, Z⁷ and Z⁸ independently are R¹⁰⁴ or L-T;
(xlv) each L is independently a direct bond or the residue of a multivalent moiety that does not significantly impair the ability of the compound to bind to a cobalamin transport protein;
(xlvi) each T is independently a diagnostic or therapeutic agent;
(xlvii) at least one of Z¹, Z², Z³, Z⁴, Z⁵, Z⁷, Z⁸, A, B, G¹, and G² comprises an a nucleic acid sequence useful in antisense technology, a peptide nucleic acid or morpholino nucleic acid;
(xlviii) J¹, J² and J³ independently are hydrogen, alkyl, alkenyl, alkynyl, alkaryl, cycloalkyl, aryl, cycloaryl, heterocycle, heteroaryl, hydroxyl, alkoxy or amine;
(xlix) R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
(l) R¹³ and R¹⁴ optionally can come together to form a pi bond; and
(li) R¹⁰⁰, R¹⁰¹, R¹⁰², R¹⁰³, and R¹⁰⁴ are independently hydrogen, alkyl, alkenyl, alkynyl, hydroxyl, alkoxy, cyano, azido, halogen, nitro, SO₂, SO₃, thioalkyl, or amino.

24. The composition of claim 19 or 23, wherein the therapeutic is an antibiotic.

25. The composition of claim 19 or 23, wherein the therapeutic is useful for the treatment of a disorder associated with abnormal cellular proliferation.

26. The composition of claim 19 or 23, wherein therapeutic agent is useful for the treatment of an infectious disease.

27. The composition of claim 19 or 23, wherein the therapeutic agent is useful in the treatment of a cardiovascular disorder.

28. The composition of claim 19 or 23, wherein the therapeutic agent is a nucleic acid, peptide nucleic acid, morpholino nucleic acid, or other material that affects gene expression.

29. The composition of claim 19 or 23, wherein the detectable agent is useful in radioimaging.

30. The composition of claim 19 or 23, wherein the detectable agent is a radionuclide or paramagnetic metal atom.

31. The composition of claim 19, wherein the cobalamin transport protein is linked directly or by a linker to a detectable radionuclide, or paramagnetic metal atom.

32. The composition of claim 22 wherein a detectable agent comprising a metallic radionuclide or paramagnetic metal atom is linked to the cobalamin.

33. The composition of claim 22 wherein the detectable chelating group is DPTA.

34. The method of claim 22 wherein the metallic radionuclide or paramagnetic metal atom is Technetium-99m, Indium-111, or Gadolinium-157.

35. The method of claim 21 wherein the detectable radionuclide is a non-metallic radionuclide.

36. The method of claim 35 wherein the non-metallic radionuclide is Carbon-11, Fluorine-18, Bromine-76, Iodine-123, or Iodine-124.


SEQUENCE LISTING (32 sequences)

SEQ ID NO: 1 - protein, 10 aa, artificial sequence
  TAT nuclear localization signal peptide
YGRKRRQRRR

SEQ ID NO: 2 - DNA, 21 bp, artificial sequence
  Vitravene (fomivirsen)
gcgtttgctcttcttcttgcg

SEQ ID NO: 3 - DNA, 20 bp, artificial sequence
  oligonucleotide- ISIS 102453
cccctcgctctccgctcccg

SEQ ID NO: 4 - DNA, 20 bp, artificial sequence
  oligonucleotide- ISIS 112043
gtgnttggctgagttnanaa

SEQ ID NO: 5 - DNA, 20 bp, artificial sequence
  oligonucleotide-- ISIS 112043
aganatttattcagcgtnan

SEQ ID NO: 6 - DNA, 20 bp, artificial sequence
  oligonucleotide-- ISIS 105990
agnaaaagatnaatnngtta

SEQ ID NO: 7 - DNA, 20 bp, artificial sequence
  oligonucleotide-- ISIS 105990
tttgntgtaattnanantga

SEQ ID NO: 8 - DNA, 20 bp, artificial sequence
  oligonucleotide-- ISIS 101528
ngngnaccgcccggcngnnc

SEQ ID NO: 9 - DNA, 20 bp, artificial sequence
  oligonucleotide-- ISIS 101528
aggangccgccatcttnnnn

SEQ ID NO: 10 - DNA, 20 bp, artificial sequence
  oligonucleotide-- ISIS 21329
gtttngcaaaatcactnggg

SEQ ID NO: 11 - DNA, 20 bp, artificial sequence
  oligonucleotide-- ISIS 21329
gggtttcgcaaaatcantng

SEQ ID NO: 12 - DNA, 18 bp, artificial sequence
  oligonucleotide-- ISIS 25237
gnnnattgctggacatgn

SEQ ID NO: 13 - DNA, 18 bp, artificial sequence
  oligonucleotide-- ISIS 25962
gtntgactttagcatntn

SEQ ID NO: 14 - DNA, 18 bp, artificial sequence
  oligonucleotide-- ISIS 25962
ggtatcttttcttctgtt

SEQ ID NO: 15 - DNA, 18 bp, artificial sequence
  oligonucleotide-- ISIS 29714
caagttgctgcctgggaa

SEQ ID NO: 16 - DNA, 18 bp, artificial sequence
  oligonucleotide-- ISIS 29714
nngacgtcttgtggatga

SEQ ID NO: 17 - DNA, 18 bp, artificial sequence
  oligonucleotide-- ISIS 29176
agtntactgctcggnnat

SEQ ID NO: 18 - DNA, 18 bp, artificial sequence
  oligonucleotide-- ISIS 29176
ntaggccccaccagtnta

SEQ ID NO: 19 - DNA, 18 bp, artificial sequence
  oligonucleotide-- ISIS 28030
atttcctgggatgtgngg

SEQ ID NO: 20 - DNA, 20 bp, artificial sequence
  oligonucleotide--  ISIS 2302
gcccaagctggcatccgtca

SEQ ID NO: 21 - DNA, 20 bp, artificial sequence
  oligonucleotide--  3521/CGP, 64128A (Isis, Novartis)
gttctcgctggtgagtttca

SEQ ID NO: 22 - DNA, 20 bp, artificial sequence
  oligonucleotide--  5132/CPG, 64128A (Isis, Novartis)
tcccgcctgtgacatgcatt

SEQ ID NO: 23 - DNA, 20 bp, artificial sequence
  oligonucleotide-- ISIS 2503
tccgtcatcgctcctcaggg

SEQ ID NO: 24 - DNA, 18 bp, artificial sequence
  oligonucleotide-- G3139 Genta
tctcccagcgtgcgccat

SEQ ID NO: 25 - DNA, 14 bp, artificial sequence
  oligonucleotide-- LR3280 Lynx
aacgttgagggcat

SEQ ID NO: 26 - DNA, 24 bp, artificial sequence
  Oligonucleotide--LR3001-Lynx
tatgctgtgccggggtcttcgggc

SEQ ID NO: 27 - DNA, 18 bp, artificial sequence
  oligonucleotide-- LR4437-Lynx
ggaccctcctccggagcc

SEQ ID NO: 28 - DNA, 20 bp, artificial sequence
  oligonucleotide-- GEM-132 (Hybridon)
uggggcttaccttgcgaaca

SEQ ID NO: 29 - DNA, 21 bp, artificial sequence
  Oligonucleotide-- GEM-92 (Hybridon)
ucgcacccatctctctccuuc

SEQ ID NO: 30 - DNA, 18 bp, ARTIFICIAL SEQUENCE
  Oligonucleotide-- GEM-231 (hybridon)
gcgugcctcctcacuggc

SEQ ID NO: 31 - DNA, 20 bp, artificial sequence
  oligonucleotide GPI-2A- Novopharm
ggttcttttggtccttgtct

SEQ ID NO: 32 - DNA, 21 bp, artificial sequence
  oligonucleotide- ISIS 13312
gcgtttgctcttcttcttgcg